(19) United States
(12) Patent Application Publication
(10) Pub. No.: US 2023/0145276 A1
(43) Pub. Date: May 11, 2023

(54) AEROSOLIZED FORMULATIONS OF AN APELIN PEPTIDE AND USES THEREOF

(71) Applicants: John CHAN, San Francisco, CA (US); Jayakumar RAJADAS, Cupertino, CA (US); Larry STEINMAN, Stanford, CA (US)

(72) Inventors: John CHAN, San Francisco, CA (US); Jayakumar RAJADAS, Cupertino, CA (US); Larry STEINMAN, Stanford, CA (US)

(21) Appl. No.: 17/914,714

(22) PCT Filed: Mar. 26, 2021
(86) PCT No.: PCT/US2021/024513

§ 371 Date: Sep. 26, 2022

Related U.S. Application Data
(60) Provisional application No. 63/001,129, filed on Mar. 27, 2020.

(51) Int. Cl. A61K 38/19; A61K 31/14; A61K 31/522; A61K 47/10; A61K 9/00

(52) U.S. Cl. CPC A61K 38/19; A61K 31/14; A61K 31/522; A61K 47/10; A61K 9/0073

(57) ABSTRACT

The present disclosure provides apelin formulations, and methods and devices of delivering apelin formulations to an individual. Also provided are methods of preventing and/or treating coronavirus-associated diseases in an individual comprising administering to the individual an effective amount of an apelin formulation comprising an apelin peptide.

### CROSS-REFERENCE TO RELATED APPLICATIONS

This application claims the priority benefit of U.S. Provisional Application No. 63/001,129, filed on Mar. 27, 2020, the contents of which are incorporated herein by reference in its entirety.

### TECHNICAL FIELD

The present disclosure is directed to apelin formulations, and methods and devices of delivering apelin formulations to an individual. Also provided are methods of preventing and/or treating coronavirus-associated diseases in an individual comprising administering to the individual an effective amount of an apelin formulation comprising an apelin peptide.

### BACKGROUND

Coronaviruses are known to cause deadly diseases. For example, Coronavirus Disease-2019 (COVID-19), which is caused by SARS-CoV-2 (also known as 2019-nCov; a member of the coronavirus family), may lead to rapid onset of Acute Respiratory Distress Syndrome (ARDS) in addition to causing cardio-pulmonary distress. COVID-19 has a high fatality rate (about 3+%). The unique and devastating characteristics of COVID-19 are attributable to the high transmissibility of SARS-CoV-2, which is akin to the common cold. SARS-CoV-2, and the spread of COVID-19, is nearly impossible to contain due to the high transmissibility and the lengthy, and often asymptomatic, incubation period (on average about 7-14 days, and up to 20+ days in certain cases). Additionally, much like influenza, coronaviruses are RNA viruses and are prone to mutation. It has already been reported that two unique strands of SARS-CoV-2 exist. Vaccine-based approaches for mitigating the spread of COVID-19 will have very limited success due the continual mutation of the virus and the 18+ month vaccine development timeline. Such a strategy will be in a “continuous” catch-up cycle. Furthermore, this viral variation will also cause certain anti-viral strategies to have very limited success. Other means for preventing and treating coronavirus-associated diseases are desperately needed.

All references cited herein, including patent applications and publications, are incorporated by reference in their entirety.

### BRIEF SUMMARY

In some aspects, provided herein is a method of preventing or treating a coronavirus-associated disease in an individual, the method comprising administering to the individual an effective amount of an apelin formulation comprising an apelin peptide.

In some embodiments, the coronavirus-associated disease is Coronavirus Disease-2019 (COVID-19), an Angiotensin-Converting Enzyme 2 (ACE2)-associated disease, Acute Respiratory Distress Syndrome (ARDS), Severe Acute Respiratory Syndrome (SARS), or Middle East respiratory syndrome (MERS).

In some embodiments, the coronavirus-associated disease causes one or more of heart failure, lung failure, or sepsis-like conditions.

In some embodiments, the coronavirus-associated disease is caused by a virus of the Coronaviridae family. In some embodiments, the virus is of the Betacoronavirus genus. In some embodiments, the virus is of the Sarbecovirus subgenus. In some embodiments, the virus is of the SARSr-CoV species. In some embodiments, the virus is a SARS-CoV strain.

In some embodiments, the virus is selected from the group consisting of Severe Acute Respiratory Syndrome coronavirus 2 (SARS-CoV-2), Severe Acute Respiratory Syndrome coronavirus (SARS-CoV), Bat SARS-like coronavirus WIV1 (Bat SL-CoV-WIV1), alpha coronaviruses 229E (HCoV-229E), New Haven coronavirus NL63 (HCoV-NL63), beta coronaviruses OC43 (HCoV-OC43), coronavirus HKU1 (HCoV-HKU1), and Middle East Respiratory Syndrome coronavirus (MERS-CoV).

In some embodiments, the method further comprises administering another therapeutic agent. In some embodiments, the other therapeutic agent is an anti-viral agent. In some embodiments, the anti-viral agent is selected from the group consisting of remdesivir, lopinavir/ritonavir, IFN-α, lopinavir, ritonavir, penciclovir, galidesivir, disulfiram, darunavir, cobicistat, ASC09F, nafamostat, griffithsin, alisporivir, chloroquine, nitazoxanide, baloxavir marboxil, oseltamivir, zanamivir, peramivir, amantadine, rimantadine, favipiravir, laninamivir, ribavirin, umifenovir, and any combination thereof.

In some embodiments, the apelin formulation is an aerosolized formulation.

In some embodiments, the apelin formulation is an intranasal apelin formulation. In some embodiments, the apelin formulation is administered to the individual via intranasal administration. In some embodiments, the apelin formulation comprises caffeine, benzalkonium chloride, and an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

In some embodiments, the apelin formulation is an inhalation apelin formulation. In some embodiments, the apelin formulation is administered to the individual via inhalation administration. In some embodiments, the apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

In some embodiments, the effective amount of the apelin peptide in the apelin formulation is about 1 mg to about 10 mg per 100 kg of body weight of the individual. In some embodiments, the apelin formulation is administered twice per week.

In some embodiments, the apelin formulation is a parenteral formulation. In some embodiments, the apelin formulation is administered to the individual via subcutaneous or intravenous administration. In some embodiments, the apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, the apelin formulation further comprises cholesterol. In some embodiments, the weight percentage of cholesterol in the liposome is about 0.1% to about 10%. In some embodiments, the effective amount of the apelin peptide in the apelin formulation is about 1 mg to about 10 mg per 100 kg of body weight of the individual. In some embodiments, the apelin formulation is administered twice per week.

In some embodiments, the at least one poloxamer is poloxamer 188, poloxamer 124, poloxamer 181, poloxamer 184, poloxamer 331, and poloxamer 407, or any combination thereof. In some embodiments, the weight percentage of the poloxamer in the liposome is about 1% to about 20%.

In some embodiments, the weight percentage of DSPC in the liposome is about 5 wt % and about 30 wt %.

In some embodiments, the weight percentage of DPPC in the liposome is about 5 wt % and about 30 wt %.

In some embodiments, the average molecular weight of the PEG is about 200 Da to about 20000 Da. In some embodiments, the average molecular weight of the PEG is about 8000 Da. In some embodiments, the weight percentage of the PEG in the liposome is about 10% to about 20%.

In some embodiments, the apelin peptide is selected from the group consisting of apelin-12, apelin-13, pyroglutamyl apelin-13 ([Pyrl]-apelin-13), apelin-17, apelin-19, and apelin-36. In some embodiments, the weight percentage of the apelin peptide in the liposome is about 15 wt % to about 60 wt %.

In some embodiments, the apelin formulation is an oral apelin formulation. In some embodiments, the apelin formulation is administered via oral administration. In some embodiments, the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the effective amount of the apelin peptide, a poloxamer, 1,2-distearoyl-sn-glycero-3-phosphocholine (DSPC), and 2-dipalmitoyl-sn-glycero-3-phosphocholine (DPPC).

In some embodiments, the lipid-based nanoparticles are liposomes comprising a lipid bilayer encapsulating a liquid core. In some embodiments, each liposome comprises a plurality of the apelin peptide, wherein a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the liquid core comprises a second subset of the plurality of the apelin peptide.

In some embodiments, the effective amount of the apelin peptide in the apelin formulation is about 1 mg to about 10 mg. In some embodiments, the apelin peptide is selected from the group consisting of apelin-12, apelin-13, pyroglutamyl apelin-13 ([Pyrl]-apelin-13), apelin-17, apelin-19, and apelin-36. In some embodiments, the apelin formulation is administered to the individual daily. In some embodiments, the weight percentage of the apelin peptide in the lipid-based nanoparticles is about 15% to about 60%.

In some embodiments, the poloxamer is poloxamer 188, poloxamer 124, poloxamer 181, poloxamer 184, poloxamer 331, and poloxamer 407, or any combination thereof. In some embodiments, the weight percentage of the poloxamer in the lipid-based nanoparticles is about 1% to about 20%.

In some embodiments, the weight percentage of DSPC in the lipid-based nanoparticles is about 5% to about 30%.

In some embodiments, the weight percentage of DPPC in the lipid-based nanoparticles is about 5% to about 30%.

In some embodiments, the lipid-based nanoparticle further comprises a polyethylene glycol (PEG). In some embodiments, the average molecular weight of the PEG is about 200 Da to about 20000 Da. In some embodiments, the average molecular weight of the PEG is about 8000 Da. In some embodiments, the weight percentage of the PEG in the lipid-based nanoparticles is about 10% to about 20%.

In some embodiments, the lipid-based nanoparticle further comprises cholesterol. In some embodiments, the weight percentage of cholesterol in the lipid-based nanoparticles is about 0.1% to about 10%.

In some embodiments, the size range of the plurality of particles is about 1 μm to about 40 μm. In some embodiments, each of the plurality of particles comprises a plurality of pores. In some embodiments, the polysaccharide is a pectin, gara gum, oak milk carbohydrate, or banana carbohydrate. In some embodiments, the pectin is a citrus peel pectin. In some embodiments, the pectin is 150-grade pectin.

In some embodiments, the cross-linking agent is selected from a divalent or polyvalent cation. In some embodiments, the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof.

In some embodiments, the apelin peptide has a bioavailability in an individual of about 2% or greater.

In other aspects, provided is an aerosolized apelin formulation comprising an effective amount of an apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, the aerosolized apelin formulation further comprises caffeine and/or benzalkonium chloride. In some embodiments, the caffeine and/or benzalkonium chloride are associated with and/or encapsulated in the liposome.

In other aspects, provided is a device for delivery of an aerosolized apelin formulation, wherein the device contains any aerosolized apelin formulation described herein, and wherein the device is configured to aerosolize the apelin formulation. In some embodiments, the device is a nasal sprayer. In some embodiments, the device is an inhaler. In some embodiments, the device is configured to provide the aerosolized apelin formulation to a ventilator.

In other aspects, provided is a method of delivering an aerosolized apelin formulation to an individual, the method comprising using any device described herein to administer the aerosolized apelin formulation to the individual in need thereof.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### DETAILED DESCRIPTION

Provided herein, in some aspects, is a method of preventing or treating a coronavirus-associated disease in an individual, the method comprising administering to the individual an effective amount of an apelin formulation comprising an apelin peptide. The present disclosure is based on the inventors' unique insights that certain apelin formulations capable of delivering an apelin peptide in vivo can be useful for preventing and/or treating a coronavirus-associated disease by enhancing the innate immune defense system, especially within the cardio-pulmonary system, reducing the ability of coronaviruses to penetrate host cells, and providing beneficial protective properties. The apelin peptide, which is produced by the human body, is a key regulator of the RAS system and provides cardio-pulmonary-metabolic protection and promotes ACE2 activity and upregulates ACE2 expression. Apelin peptides have shown both safety and efficacy in clinical studies involving patients with heart failure, pulmonary hypertension, and insulin insensitivity. Apelin also provides broad, systemic anti-inflammatory protection as demonstrated in a sepsis model. The level of endogenous apelin peptides decreases with age and presentation of chronic diseases, such as cardiovascular disease and diabetes.

As disclosed herein, the inventors provide apelin formulations for use in a method of preventing or treating a coronavirus-associated disease, such as COVID-19. Use of such apelin formulations will enhance the RAS function and the immunity of the individual, providing the subject with greater resistance against infection by a coronavirus. Use of such apelin formulations will also increase ACE2 expression that will inhibit the downstream acute respiratory distress (and/or heart failure) that, e.g., occurs during COVID-19, and will also provide broad anti-inflammatory protection against the documented, over-active inflammatory response “cytokine storm” as observed among COVID-19 patients. The apelin formulations described herein provide apelin with a high bioavailability and cellular uptake.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

The term “peptide,” such as used in the phrase “apelin peptide,” refers to a polymer comprising amino acid residues, and is not to be construed as implying a limitation regarding the number of amino acids and/or length thereof. Such polymers may contain natural amino acids and/or or non-natural amino acid. In some embodiments, the term “polypeptide” also encompasses modified species of polypeptides, e.g., polypeptides comprising one or more chemical modifications and/or one or more post-translational modifications.

The term “sequence identity,” with respect to a polypeptide or peptide comprising an amino acid sequence refers to the percentage of amino acid residues in a candidate sequence that are identical to the amino acid residues in the specific protein or amino acid sequence after aligning the sequences and introducing gaps, if necessary, to achieve a maximum percent sequence identity, and not considering any conservative substitutions as part of the sequence identity. Alignment can be achieved by any method known to one of skill in the art, for example, by using publicly available programs such as BLAST and EIBOSS. Those skilled in the art can determine appropriate parameters for measuring alignment, including any algorithms needed to achieve maximal alignment over the full length of the sequences being compared.

As used herein, the terms “treating” or “preventing,” or grammatical equivalents thereof, encompass approaches for obtaining or maintaining beneficial or desired results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the disease, preventing or delaying the spread of the disease, preventing or delaying the recurrence of the disease, delaying or slowing the progression of the disease, ameliorating the disease state, providing a remission (e.g., partial or total) of the disease, delaying the progression of the disease, increasing the quality of life, and/or prolonging survival. The methods of the present application contemplate any one or more of these aspects of treatment.

The term “effective amount,” as used herein, refers to an amount of a compound or composition sufficient to treat or prevent a specified disorder, condition, or disease such as ameliorate, palliate, lessen, and/or delay one or more of its symptoms. As is understood in the art, an “effective amount” may be in one or more doses, e.g., a single dose or multiple doses may be required to achieve the desired treatment endpoint. An effective amount may be considered in the context of administering one or more therapeutic agents, and combinations may be considered to be given in an effective amount if a desirable or beneficial result may be or is achieved. The components (e.g., the first and second therapies) in a combination treatment described in the present application may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

The term “individual” refers to a mammal and includes, but is not limited to, human, bovine, horse, feline, canine, rodent, or primate.

The term “pharmaceutically acceptable,” as used herein, is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers, excipients, or salts have preferably met the required standards of toxicological and manufacturing testing and/or are included on the Inactive Ingredient Guide prepared by the U.S. Food and Drug administration.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.” In some embodiments, numerical designations are provided herein for ease of understanding the scope of the present disclosure, wherein the numerical designations are calculated from experimental values and may include approximations, e.g., rounded weight percentages calculated from an amount of a starting material. In some embodiments, numerical designations provided herein, e.g., weight percentages, may vary (±) by increments of 0.1 to 0.5.

As used herein, including in the appended claims, the singular forms “a,” “or,” and “the” include plural referents unless the context clearly dictates otherwise.

### A. Methods of Treatment

Provided herein, in some aspects, is a method of preventing or treating a coronavirus-associated disease in an individual, the method comprising administering to the individual an effective amount of an apelin formulation comprising an apelin peptide. In some embodiments, described herein is a method of preventing. In some embodiments, described herein is a method of treating.

In some embodiments, the coronavirus-associated disease is Coronavirus Disease-2019 (COVID-19), an Angiotensin-Converting Enzyme 2 (ACE2)-associated disease, Acute Respiratory Distress Syndrome (ARDS), Severe Acute Respiratory Syndrome (SARS), or Middle East respiratory syndrome (MERS). In some embodiments, the ACE2-associated disease is a disease, such as a viral infection, that causes a modulation in the cell-surface level, such as a decrease, of ACE2. In some embodiments, the ACE2-associated disease is a disease, such as a viral infection, that causes a modulation in the serum level, such as an increase, of ACE2.

In some embodiments, the coronavirus-associated disease causes cardio-pulmonary distress, including increased breathing rate, increased heart rate, and modulation of blood pressure.

In some embodiments, the coronavirus-associated disease causes one or more of heart failure, lung failure, or sepsis-like conditions. In some embodiments, the coronavirus-associated disease causes heart failure. In some embodiments, the coronavirus-associated disease causes lung failure. In some embodiments, the coronavirus-associated disease causes sepsis-like conditions. In some embodiments, the coronavirus-associated disease causes heart failure and lung failure. In some embodiments, the coronavirus-associated disease causes heart failure and sepsis-like conditions. In some embodiments, the coronavirus-associated disease causes lung failure and sepsis-like conditions. In some embodiments, the coronavirus-associated disease causes heart failure, lung failure, and sepsis-like conditions. In some embodiments, the coronavirus-associated disease causes pulmonary hypertension.

In some embodiments, the coronavirus-associated disease is Coronavirus Disease-2019 (COVID-19), Acute Respiratory Distress Syndrome (ARDS), Severe Acute Respiratory Syndrome (SARS), or Middle East respiratory syndrome (MERS).

In some embodiments, the coronavirus-associated disease is caused by a virus of the Coronaviridae family. In some embodiments, the virus is of the Betacoronavirus genus. In some embodiments, the virus is of the Sarbecovirus subgenus. In some embodiments, the virus is of the SARSr-CoV species. In some embodiments, the virus is a SARS-CoV strain.

In some embodiments, the virus is selected from the group consisting of Severe Acute Respiratory Syndrome coronavirus 2 (SARS-CoV-2), Severe Acute Respiratory Syndrome coronavirus (SARS-CoV), Bat SARS-like coronavirus WIV1 (Bat SL-CoV-WIV1), alpha coronaviruses 229E (HCoV-229E), New Haven coronavirus NL63 (HCoV-NL63), beta coronaviruses OC43 (HCoV-OC43), coronavirus HKU1 (HCoV-HKU1), and Middle East Respiratory Syndrome coronavirus (MERS-CoV).

The methods described herein comprise administering an effective amount of an apelin formulation comprising an apelin peptide. In any of the embodiments described herein, the apelin peptide is selected from the group consisting of apelin-12, apelin-13, pyroglutamyl apelin-13 ([Pyrl]-apelin-13), apelin-17, apelin-19, and apelin-36. In some embodiments, the apelin peptide is pyroglutamyl apelin-13 ([Pyrl]-apelin-13).

Apelin peptides, and biologically active variants, within the scope of the present disclosure are described in U.S. PG Patent Publication. No. 2016/0058705, which is incorporated herein by reference in its entirety. In some embodiments, the apelin peptide comprises an amino acid sequence selected from the group consisting of SEQ ID NOS: 1-7 (Table 1). In some embodiments, the apelin peptide comprises a sequence having at least about any of 60%, 65%, 70%, 75%, 80%, 85%, 90%, 95%, 96%, 97%, 98%, or 99% sequence identity to a sequence from SEQ ID NOS:1-7. In some embodiments, the apelin peptide, such as any apelin peptide from SEQ ID NOS: 1-7, comprises one or more, such as any of 2, 3, 4, or 5, amino acid changes selected from any one or more of an addition, substitution, and/or deletion. In some embodiments, the apelin peptide comprises a modification, such as a post-translation modification.

TABLE 1
Apelin peptide sequences.

| SEQ | | |
|---|---|---|
| ID NO. | Sequence | Name |
| 1 | Met Asn Leu Arg Leu Cys Val Gln | Apelin |
| | Ala Leu Leu Leu Leu Trp Leu Ser | preprotein |
| | Leu Thr Ala Val Cys Gly Gly Ser | |
| | Leu Met Pro Leu Pro Asp Gly Asn | |
| | Gly Leu Glu Asp Gly Asn Val Arg | |
| | His Leu Val Gln Pro Arg Gly Ser | |
| | Arg Asn Gly Pro Gly Pro Trp Gln | |
| | Gly Gly Arg Arg Lys Phe Arg Arg | |
| | Gln Arg Pro Arg Leu Ser His Lys | |
| | Gly Pro Met Pro Phe | |
| 2 | Arg Pro Arg Leu Ser His Lys Gly | Apelin-12 |
| | Pro Met Pro Phe | |
| 3 | Gln Arg Pro Arg Leu Ser His Lys | Apelin-13 |
| | Gly Pro Met Pro Phe | |
| 4 | Xaa Arg Pro Arg Leu Ser His Lys | [Pyr1]- |
| | Gly Pro Met Pro Phe | apelin-13 |
| | (Xaa/X is pyroglutamate) | |
| 5 | Lys Phe Arg Arg Gln Arg Pro Arg | Apelin-17 |
| | Leu Ser His Lys Gly Pro Met Pro | |
| | Phe | |
| 6 | Arg Arg Lys Phe Arg Arg Gln Arg | Apelin-19 |
| | Pro Arg Leu Ser His Lys Gly Pro | |
| | Met Pro Phe | |
| 7 | Leu Val Gln Pro Arg Gly Ser Arg | Apelin-36 |
| | Asn Gly Pro Gly Pro Trp Gln Gly | |
| | Gly Arg Arg Lys Phe Arg Arg Gln | |
| | Arg Pro Arg Leu Ser His Lys Gly | |
| | Pro Met Pro Phe | |

The methods described herein provide delivery of an apelin peptide using aerosolized formulations, parenteral formulations, and oral formulations. As described in more detail in the sections below, the apelin formulations described herein encompass a range of working component weight percentages. One of ordinary skill in the art will readily recognize that descriptions using weight percentages are based on the components included in the total weight used in the weight percentage calculation. For example, adding and/or subtracting one or more additional components to an oral formulations described herein will adjust the weight percentages of the other components of the oral formulation if included in the total weight used in the weight percentage calculation. Thus, in some embodiments, weight percentages are provided relative to a list of one or more provided components used to calculate the total weight used in the weight percentage calculation.

In some embodiments, the apelin formulations provided herein comprise one or more pharmaceutically acceptable agent, such as any of an excipient, salt, diluent, carrier, vehicle, bulking agent, other inactive agents used to formulate formulations, or any combination thereof. Vehicles and excipients commonly employed in pharmaceutically acceptable formulations include, for example, talc, gum Arabic, lactose, starch, magnesium stearate, cocoa butter, aqueous or non-aqueous solvents, oils, paraffin derivatives, glycols, etc. Formulations in solution can be prepared using water or physiologically compatible organic solvents such as ethanol, 1,2-propylene glycol, polyglycols, dimethylsulfoxide, fatty alcohols, triglycerides, partial esters of glycerine and the like. Formulations may be prepared using conventional techniques that may include sterile isotonic saline, water, 1,3-butanediol, ethanol, 1,2-propylene glycol, polyglycols mixed with water, and Ringer's solution. In some embodiment, the formulation comprises a coloring agent to facilitate in locating and properly placing the formulation at the intended treatment site. Vehicles and excipients commonly employed in oral dosage forms include, for example, talc, gum Arabic, lactose, starch, magnesium stearate, cocoa butter, and paraffin derivatives. In some embodiments, the oral dosage form further comprises a preservative and/or a stabilizer. In some embodiments, the oral dosage form further comprises a cryoprotectant agent. In some embodiments, the apelin formulation is a sterile apelin formulation.

In some embodiments, the apelin formulation is soluble in the pharmaceutically acceptable excipient. In some embodiments, the apelin formulation is soluble in, and does not precipitate out of, the pharmaceutically acceptable excipient for a period of time from about 30 minutes, about 1 hour, about 2 hours, about 3 hours, about 4 hours, about 5 hours, about 6 hours, about 12 hours, about 1 day, about 2 days, about 3 days, about 4 days, about 5 day, about 6 days, about 1 week, about 2 weeks, about 3 weeks, about a month, or longer.

### 1. Aerosolized Formulations of Apelin for Intranasal and Inhalation Administration

In some embodiments, provided herein is a method of preventing or treating a coronavirus-associated disease in an individual, the method comprising administering to the individual an effective amount of an aerosolized apelin formulation comprising an apelin peptide. Also provided are aerosolized apelin formulations, and methods and devices for delivery an aerosolized apelin formulation to an individual in need thereof.

In some embodiments, the effective amount of the apelin peptide in the aerosolized apelin formulation is about 1 mg to about 10 mg, such as any of about 1 mg to about 8 mg, about 1 mg to about 5 mg, about 3 mg to about 8 mg, about 2 mg to about 6 mg, or about 3 mg to about 5 mg, per 100 kg of body weight of the individual. In some embodiments, the effective amount of the apelin peptide in the aerosolized apelin formulation is at least about 0.5 mg, such as at least about any of 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, per 100 kg of body weight of the individual. In some embodiments, the effective amount of the apelin peptide in the aerosolized apelin formulation is less than about 10 mg, such as less than about any of 9.5 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.5 mg, 2 mg, 1.5 mg, or 1 mg, per 100 kg of body weight of the individual. In some embodiments, the effective amount of the apelin peptide in the aerosolized apelin formulation is about any of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, per 100 kg of body weight of the individual.

In some embodiments, the aerosolized apelin formulation comprises an effective amount of the apelin peptide that is at least partially encapsulated in a liposome. In some embodiments, the liposomes comprise a lipid bilayer encapsulating a liquid core. In some embodiments, wherein each liposome comprises a plurality of the apelin peptide, a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the liposomes are configured such that the apelin peptide presented on the outer surface of the liposomes may associate with, such as bind, a relevant receptor and/or target binding site. In some embodiments, the aerosolized apelin formulation comprises an amount of an apelin peptide that is not associated with or encapsulated in a liposome.

In some embodiments, the liposomes comprises a liquid core comprising a second subset of the plurality of the apelin peptide. In some embodiments, the liposomes are configured to hold a certain concentration, or range thereof, of the second subset of the apelin peptide.

Although an apelin peptide is exemplified throughout the aerosolized apelin formulations of the present disclosure, the description encompasses use of a biologically active peptide for any of the aerosolized formulation embodiments described herein. In some embodiments, the biologically active peptide comprises a stretch of at least about 15, such as at least about any of 20, 25, or 30, contiguous amino acids having a net hydrophobic characteristic. In some embodiments, the biologically active peptide comprises a stretch of at least about 15, such as at least about any of 20, 25, or 30, contiguous amino acids, wherein the stretch comprises more hydrophobic amino acid residues than hydrophilic amino acid residues. In some embodiments, the biologically active peptide comprises a stretch of at least about 15, such as at least about any of 20, 25, or 30, contiguous amino acids, wherein the stretch comprises at least about 55%, such as at least about any of 60%, 65%, 70%, 75%, 80%, 85%, 90%, 95%, or 100%, hydrophobic amino acid residues. One of ordinary skill in the art will readily understand and be able to identify hydrophobic amino acids, e.g., glycine, alanine, valine, leucine, isoleucine, proline, phenylalanine, methionine, and tryptophan.

In some embodiments, the biologically active peptide comprises a stretch of at least about 10, such as at least about any of 15, 20, 25, or 30, contiguous amino acids having a net positive charge at pH 7. In some embodiments, the biologically active peptide comprises a stretch of at least about 10, such as at least about any of 15, 20, 25, or 30, contiguous amino acids having a net positive charge at a physiological pH of the gastrointestinal tract. One of ordinary skill in the art will readily understand and be able to identify charged amino acids and the impact of pH on the charge of an amino acid, e.g., lysine and arginine.

In some embodiments, the biologically active peptide comprises a stretch of at least about 15, such as at least about any of 20, 25, or 30, contiguous amino acids having a net hydrophobic characteristic at or near, such as within about 5 amino acids, the N-terminus of the biologically active peptide. In some embodiments, the biologically active peptide comprises a stretch of at least about 10, such as at least about any of 15, 20, 25, or 30, contiguous amino acids having a net positive charge at pH 7 at or near, such as within about 5 amino acids, of the C-terminus. In some embodiments, the biologically active peptide comprises, from N- to C-terminus, the stretch of amino acids having a net hydrophobic characteristic and the stretch of amino acids having a net positive charge. In some embodiments, the biologically active peptide comprises one or more stretches of other amino acids between the stretch of amino acids having a net hydrophobic characteristic and the stretch of amino acids having a net positive charge. In some embodiments, the biologically active peptide comprises, from N- to C-terminus, a stretch of amino acids having a net hydrophobic characteristic, a stretch of at least about 5, such as at least about any of 10, 15, 20, 25, 30, 35, or 40, amino acids, and a stretch of amino acids having a net positive charge.

The aerosolized apelin formulation can be administered according to the discretion of the practicing physician. In some embodiments, the aerosolized apelin formulation is administered daily. In some embodiments, the aerosolized apelin formulation is administered one or more, such as any of 2, 3, 4, 5, or 6, times per week. In some embodiments, the aerosolized apelin formulation is administered twice per week. In some embodiments, the aerosolized apelin formulation is administered weekly.

Administration of aerosolized formulations are known in the art, and include intranasal administration and inhalation administration, such as via a nasal spray, nebulizer, or an inhaler, such as a metered dose inhaler or pressurized metered dose inhaler. In some embodiments, the aerosolized formulation is administered via a mechanical ventilator. In some embodiments, the aerosolized apelin formulation is an intranasal formulation. In some embodiments, the aerosolized apelin formulation is an inhalation formulation.

i. Intranasal Formulation and Administration Thereof

In some embodiments, the apelin formulation is an intranasal apelin formulation. In some embodiments, the apelin formulation is administered to the individual via intranasal administration, such as by using a nasal sprayer or nebulizer. In some embodiments, the intranasal apelin formulation is a dry formulation. In some embodiments, the intranasal apelin formulation is a wet formulation.

In some embodiments, the apelin formulation is suitable for intranasal administration. In some embodiments, the intranasal apelin formulation comprises caffeine, benzalkonium chloride, and an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, the benzalkonium chloride is associated with and/or encapsulated in the liposome. In some embodiments, the liposomes comprise a lipid bilayer encapsulating a liquid core. In some embodiments, wherein each liposome comprises a plurality of the apelin peptide, a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the liposomes are configured such that the apelin peptide presented on the outer surface of the liposomes may associate with, such as bind, a relevant receptor and/or target binding site. In some embodiments, the intranasal apelin formulation comprises an amount of an apelin peptide that is not associated with or encapsulated in a liposome.

The intranasal apelin formulations described herein comprise an apelin peptide. In some embodiments, the weight percentages of apelin in the intranasal apelin formulation is about 5% to about 60%, such as about 15% to about 20% or about 5% to about 45%. In some embodiments, the weight percentages of apelin in the intranasal apelin formulation is at least about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, the weight percentages of apelin in the intranasal apelin formulation is at less than about any 60%, 59%, 58%, 57%, 56%, 55%, 54%, 53%, 52%, 51%, 50%, 49%, 48%, 47%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 19%, 18%, 17%, 16%, 15%, 14%, 13%, 12%, 11%, 10%, 9%, 8%, 7%, 6%, or 5%. In some embodiments, the weight percentages of apelin in the intranasal apelin formulation is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%.

The intranasal apelin formulations described herein comprise caffeine. In some embodiments, caffeine is admixed with the liposomes in the intranasal apelin formulation. In some embodiments, caffeine is associated with and/or encapsulated in the liposomes in the intranasal apelin formulation. In some embodiments, caffeine is admixed with the liposomes in the intranasal apelin formulation and caffeine is associated with and/or encapsulated in the liposomes in the intranasal apelin formulation. In some embodiments, the weight percentage of caffeine in the intranasal apelin formulation is at least about 0.001%, such as at least about any of 0.005%, 0.01%, 0.015%, 0.02% or 0.025%. In some embodiments, the weight percentage of caffeine in the intranasal apelin formulation is about any of 0.001%, 0.005%, 0.01%, 0.015%, 0.02% or 0.025%.

The intranasal apelin formulations described herein comprise benzalkonium chloride. In some embodiments, benzalkonium chloride is admixed with the liposomes in the intranasal apelin formulation. In some embodiments, benzalkonium chloride is associated with and/or encapsulated in the liposomes in the intranasal apelin formulation. In some embodiments, benzalkonium chloride is admixed with the liposomes in the intranasal apelin formulation and benzalkonium chloride is associated with and/or encapsulated in the liposomes in the intranasal apelin formulation. In some embodiments, the weight percentage of benzalkonium chloride in the intranasal apelin formulation is at least about 0.001%, such as at least about any of 0.005%, 0.01%, 0.015%, 0.02% or 0.025%. In some embodiments, the weight percentage of benzalkonium chloride in the intranasal apelin formulation is about any of 0.001%, 0.005%, 0.01%, 0.015%, 0.02% or 0.025%.

The liposomes in the intranasal apelin formulations described herein comprise at least one poloxamer. In some embodiments, the at least one poloxamer is one or more of poloxamer 188, poloxamer 124, poloxamer 181, poloxamer 184, poloxamer 331, and poloxamer 407, or any combination thereof. In some embodiments, the poloxamer is poloxamer 188.

In some embodiments, the weight percentage of the poloxamer in the liposome is between about 1% and about 20%, such as between any of about 1% and about 18%, about 2% and about 14%, about 5% and about 11%, about 8% and about 9%, about 7.3% and about 9.3%, about 10% and about 20%, about 17.5% and about 22.5%, or about 14% and about 16%. In some embodiments, the weight percentage of the poloxamer in the liposome is at least about 1%, such as at least about any of 2%, 3%, 4%, 5%, 6%, 7%, 8%, 9%, 10%, 11%, 12%, 13%, 14%, 15%, 16%, 17%, 18%, 19%, or 20%. In some embodiments, the weight percentage of the poloxamer in the liposome is about 20% or less, such as about any of 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less. In some embodiments, the weight percentage of the poloxamer in the liposome is about any of 1%, 2%, 3%, 4%, 5%, 6%, 7%, 8%, 8.3%, 9%, 10%, 11%, 12%, 13%, 14%, 15%, 16%, 17%, 18%, 19%, 20%, 21%, 22%, 23%, 24%, or 25%.

The liposomes in the intranasal apelin formulations described herein comprise 1,2-distearoyl-sn-glycero-3-phosphocholine (DSPC). In some embodiments, the weight percentage of DSPC in liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DSPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DSPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DSPC in the liposome is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The liposomes in the intranasal apelin formulations described herein comprise 2-dipalmitoyl-sn-glycero-3-phosphocholine (DPPC). In some embodiments, the weight percentage of DPPC in the liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DPPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DPPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DPPC in the liposome is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The liposomes in the intranasal apelin formulations described herein comprise polyethylene glycol (PEG). In some embodiments, the PEG has an average molecular weight of between about 200 to about 20,000 Daltons. In some embodiments, the PEG is PEG 200, PEG 300, PEG 400, PEG 1000, PEG 1540, PEG 4000, PEG 5000, PEG 6000, PEG 7000, PEG 8000, PEG 9000, or PEG 10000. In some embodiments, the PEG is PEG 8000.

In some embodiments, the weight percentage of the PEG in the liposome is between about 10% and about 20%, such as between any of about 12.5% and about 17.5%, about 14% and about 16%, about 15.6% and about 17.6%. In some embodiments, the weight percentage of the PEG in the liposome is at least about 10%, such as at least about any of 11%, 12%, 13%, 14%, 15%, 16%, 17%, 18%, 19%, or 20%. In some embodiments, the weight percentage of the PEG in the liposome is about 20% or less, such as about any of 19% or less, 18% or less, 17% or less, 16% or less, 15% or less, 14% or less, 13% or less, 12% or less, 11% or less, or 10% or less. In some embodiments, the weight percentage of the PEG in the liposome is about any of 10%, 11%, 12%, 13%, 14%, 15%, 16%, 16.6%, 17%, 18%, 19%, or 20%.

In some embodiments, the liposomes in the intranasal apelin formulations described herein optionally comprise cholesterol. In some embodiments, the weight percentage of cholesterol in the liposome is between about 0.1% and about 10%, such as between any of about 2.5% and about 7.5%, or about 4% and about 6%.

In some embodiments, the weight percentage of cholesterol in the liposome is at least about 0.1%, such as at least about any of 1%, 2%, 3%, 4%, 5%, 6%, 7%, 8%, 9%, or 10%. In some embodiments, the weight percentage of cholesterol in the liposome is about 10% or less, such as about any of 9% or less, 8% or less, 7% or less, 6% or less, 5% or less, 4% or less, 3% or less, 2% or less, or 1% or less. In some embodiments, the weight percentage of cholesterol in the liposome is about any of 1%, 2%, 3%, 4%, 5%, 6%, 7%, 8%, 9%, or 10%.

In some embodiments, the intranasal apelin formulations described herein comprise a weight percentage of the apelin peptide of between about 15.5% and 17.5%, a weight percentage of the poloxamer (e.g., poloxamer 188) of between about 8.3% and 10.3%, a weight percentage of DSPC of between about 27% and about 29%, a weight percentage of DPPC of between about 27% and about 29%, a weight percentage of the PEG (e.g., PEG 8000) of between about 17.5% and about 19.5%, a weight percentage of caffeine of between about 0.01% and 0.3%, and a weight percentage of benzalkonium chloride of about 0.0001% and 0.01%, wherein the liposomes of the intranasal apelin formulation comprise at least the apelin peptide, the DSPC, DPPC, and the PEG (e.g., PEG 8000).

In some embodiments, the intranasal apelin formulations described herein comprise a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.510%, a weight percentage of caffeine of about 0.019%, and a weight percentage of benzalkonium chloride of about 0.009%, wherein the liposomes of the intranasal apelin formulation comprise at least the apelin peptide, the DSPC, DPPC, and the PEG (e.g., PEG 8000).

In some embodiments, the liposomes in the intranasal apelin formulations described herein comprise a weight percentage of the apelin peptide of between about 15.5% and 17.5%, a weight percentage of the poloxamer (e.g., poloxamer 188) of between about 8.3% and 10.3%, a weight percentage of DSPC of between about 27% and about 29%, a weight percentage of DPPC of between about 27% and about 29%, a weight percentage of the PEG (e.g., PEG 8000) of between about 17.5% and about 19.5%, a weight percentage of caffeine of between about 0.01% and 0.3%, and a weight percentage of benzalkonium chloride of about 0.0001% and 0.01%.

In some embodiments, the liposomes in the intranasal apelin formulations described herein comprise a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.51%, a weight percentage of caffeine of about 0.019%, and a weight percentage of benzalkonium chloride of about 0.009%.

In some embodiments, the liposomes in the intranasal apelin formulations described herein are prepared according to Formulation 1 (Table 2).

TABLE 2
Formulation 1.

Formulation 1

| | Weight (mg) | Approx. Weight % |
|---|---|---|
| DSPC | 300 | 27.77 |
| DPPC | 300 | 27.77 |
| Poloxamer 188 | 100 | 9.26 |
| PEG 8000 | 200 | 18.51 |
| [Pyr1]-Apelin-13 | 180 | 16.66 |
| Caffeine | 0.2 | 0.019 |
| Benzalkonium chloride | 0.1 | 0.009 |
| TOTAL | 1080.3 | 100.00% |

Methods of making intranasal apelin formulations and liposomes described herein are known in the art. In some embodiments, the liposomes are made by admixing a poloxamer, DSPC, DPPC, a PEG, and optionally cholesterol, to form a lipid film. In some embodiments, caffeine and/or benzalkonium chloride are added to form the lipid film. In some embodiments, caffeine and/or benzalkonium chloride are added after lipid film formation. The apelin peptide is then slowly added to the lipid film, thereby forming the liposomes. In some embodiments, caffeine and/or benzalkonium chloride are added after liposome formation. See, e.g., International application publication WO2018075822, which is hereby incorporated herein in its entirety.

In some embodiments, provided herein is a method of preventing or treating a coronavirus-associated disease in an individual, the method comprising administering to the individual an effective amount of an intranasal apelin formulation comprising an apelin peptide, wherein the intranasal apelin formulation comprises a weight percentage of the apelin peptide of between about 15.5% and 17.5%, a weight percentage of the poloxamer (e.g., poloxamer 188) of between about 8.3% and 10.3%, a weight percentage of DSPC of between about 27% and about 29%, a weight percentage of DPPC of between about 27% and about 29%, a weight percentage of the PEG (e.g., PEG 8000) of between about 17.5% and about 19.5%, a weight percentage of caffeine of between about 0.01% and 0.3%, and a weight percentage of benzalkonium chloride of about 0.0001% and 0.01%, wherein the liposomes of the intranasal apelin formulation comprise at least the apelin peptide, the DSPC, DPPC, and the PEG (e.g., PEG 8000), and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an intranasal apelin formulation comprising an apelin peptide, wherein the intranasal apelin formulations comprises a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.510%, a weight percentage of caffeine of about 0.019%, and a weight percentage of benzalkonium chloride of about 0.009%, wherein the liposomes of the intranasal apelin formulation comprise at least the apelin peptide, the DSPC, DPPC, and the PEG (e.g., PEG 8000), and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an intranasal apelin formulation comprising an apelin peptide, wherein the liposomes in the intranasal apelin formulations comprise a weight percentage of the apelin peptide of between about 15.5% and 17.5%, a weight percentage of the poloxamer (e.g., poloxamer 188) of between about 8.3% and 10.3%, a weight percentage of DSPC of between about 27% and about 29%, a weight percentage of DPPC of between about 27% and about 29%, a weight percentage of the PEG (e.g., PEG 8000) of between about 17.5% and about 19.5%, a weight percentage of caffeine of between about 0.01% and 0.3%, and a weight percentage of benzalkonium chloride of about 0.0001% and 0.01%, and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an intranasal apelin formulation comprising an apelin peptide, wherein the liposomes in the intranasal apelin formulations comprise a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.51%, a weight percentage of caffeine of about 0.019%, and a weight percentage of benzalkonium chloride of about 0.009%, and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

ii. Inhalation Formulation and Administration Thereof

In some embodiments, the apelin formulation is an inhalation apelin formulation. In some embodiments, the apelin formulation is administered to the individual via inhalation administration, such as by using an inhaler, e.g., a metered dose inhaler. In some embodiments, the inhalation apelin formulation is a dry formulation. In some embodiments, the inhalation apelin formulation is a wet formulation.

In some embodiments, the apelin formulation is suitable for inhalation administration. In some embodiments, the inhalation apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, the liposomes comprise a lipid bilayer encapsulating a liquid core. In some embodiments, wherein each liposome comprises a plurality of the apelin peptide, a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the liposomes are configured such that the apelin peptide presented on the outer surface of the liposomes may associate with, such as bind, a relevant receptor and/or target binding site. In some embodiments, the inhalation apelin formulation comprises an amount of an apelin peptide that is not associated with or encapsulated in a liposome.

The inhalation apelin formulations described herein comprise an apelin peptide. In some embodiments, the weight percentages of apelin in the inhalation apelin formulation is about 5% to about 60%, such as about 15% to about 20% or about 5% to about 45%. In some embodiments, the weight percentages of apelin in the inhalation apelin formulation is at least about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%. In some embodiments, the weight percentages of apelin in the inhalation apelin formulation is at less than about any 60%, 59%, 58%, 57%, 56%, 55%, 54%, 53%, 52%, 51%, 50%, 49%, 48%, 47%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 19%, 18%, 17%, 16%, 15%, 14%, 13%, 12%, 11%, 10%, 9%, 8%, 7%, 6%, or 5%. In some embodiments, the weight percentages of apelin in the inhalation apelin formulation is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60%.

The liposomes in the inhalation apelin formulations described herein comprise at least one poloxamer. In some embodiments, the at least one poloxamer is one or more of poloxamer 188, poloxamer 124, poloxamer 181, poloxamer 184, poloxamer 331, and poloxamer 407, or any combination thereof. In some embodiments, the poloxamer is poloxamer 188.

In some embodiments, the weight percentage of the poloxamer in the liposome is between about 1% and about 20%, such as between any of about 1% and about 18%, about 2% and about 14%, about 5% and about 11%, about 8% and about 9%, about 7.3% and about 9.3%, about 10% and about 20%, about 17.5% and about 22.5%, or about 14% and about 16%. In some embodiments, the weight percentage of the poloxamer in the liposome is at least about 1%, such as at least about any of 2%, 3%, 4%, 5%, 6%, 7%, 8%, 9%, 10%, 11%, 12%, 13%, 14%, 15%, 16%, 17%, 18%, 19%, or 20%. In some embodiments, the weight percentage of the poloxamer in the liposome is about 20% or less, such as about any of 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less. In some embodiments, the weight percentage of the poloxamer in the liposome is about any of 1%, 2%, 3%, 4%, 5%, 6%, 7%, 8%, 8.3%, 9%, 10%, 11%, 12%, 13%, 14%, 15%, 16%, 17%, 18%, 19%, 20%, 21%, 22%, 23%, 24%, or 25%.

The liposomes in the inhalation apelin formulations described herein comprise 1,2-distearoyl-sn-glycero-3-phosphocholine (DSPC). In some embodiments, the weight percentage of DSPC in liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DSPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DSPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DSPC in the liposome is about any of 5%, 6%, 7%, 8%, 9%, 10%, 11%, 12%, 13%, 14%, 16%, 17%, 18%, 19%, 20%, 21%, 22%, 23%, 24%, 25%, 26%, 27%, 28%, 29%, or 30%.

The liposomes in the inhalation apelin formulations described herein comprise 2-dipalmitoyl-sn-glycero-3-phosphocholine (DPPC). In some embodiments, the weight percentage of DPPC in the liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DPPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DPPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DPPC in the liposome is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The liposomes in the inhalation apelin formulations described herein comprise polyethylene glycol (PEG). In some embodiments, the PEG has an average molecular weight of between about 200 to about 20,000 Daltons. In some embodiments, the PEG is PEG 200, PEG 300, PEG 400, PEG 1000, PEG 1540, PEG 4000, PEG 5000, PEG 6000, PEG 7000, PEG 8000, PEG 9000, or PEG 10000. In some embodiments, the PEG is PEG 8000.

In some embodiments, the weight percentage of the PEG in the liposome is between about 10% and about 20%, such as between any of about 12.5% and about 17.5%, about 14% and about 16%, about 15.6% and about 17.6%. In some embodiments, the weight percentage of the PEG in the liposome is at least about 10%, such as at least about any of 11%, 12%, 13%, 14%, 15%, 16%, 17%, 18%, 19%, or 20%. In some embodiments, the weight percentage of the PEG in the liposome is about 20% or less, such as about any of 19% or less, 18% or less, 17% or less, 16% or less, 15% or less, 14% or less, 13% or less, 12% or less, 11% or less, or 10% or less. In some embodiments, the weight percentage of the PEG in the liposome is about any of 10%, 11%, 12%, 13%, 14%, 15%, 16%, 16.6%, 17%, 18%, 19%, or 20%.

In some embodiments, the liposomes in the inhalation apelin formulations described herein optionally comprise cholesterol. In some embodiments, the weight percentage of cholesterol in the liposome is between about 0.1% and about 10%, such as between any of about 2.5% and about 7.5%, or about 4% and about 6%.

In some embodiments, the weight percentage of cholesterol in the liposome is at least about 0.1, such as at least about any of 1%, 2%, 3%, 4%, 5%, 6%, 7%, 8%, 9%, or 10%. In some embodiments, the weight percentage of cholesterol in the liposome is about 10% or less, such as about any of 9% or less, 8% or less, 7% or less, 6% or less, 5% or less, 4% or less, 3% or less, 2% or less, or 1% or less. In some embodiments, the weight percentage of cholesterol in the liposome is about any of 1%, 2%, 3%, 4%, 5%, 6%, 7%, 8%, 9%, or 10%.

In some embodiments, the liposomes in the inhalation apelin formulations described herein comprise a weight percentage of the apelin peptide of between about 15.7% and 17.7%, a weight percentage of the poloxamer (e.g., poloxamer 188) of between about 8.3% and 10.3%, a weight percentage of DSPC of between about 27% and about 29%, a weight percentage of DPPC of between about 27% and about 29%, and a weight percentage of the PEG (e.g., PEG 8000) of between about 17.5% and about 19.5%.

In some embodiments, the liposomes in the inhalation apelin formulations described herein comprise a weight percentage of the apelin peptide of about 16.67%, a weight percentage of poloxamer 188 of about 9.25%, a weight percentage of DSPC of about 27.77%, a weight percentage of DPPC of about 27.77%, and a weight percentage of PEG 8000 of about 18.52%.

In some embodiments, the liposomes in the inhalation apelin formulations described herein are prepared according to Formulation 1 (Table 3).

TABLE 3
Formulation 2.

Formulation 2

| | Weight (mg) | Approx. Weight % |
|---|---|---|
| DSPC | 300 | 27.77 |
| DPPC | 300 | 27.77 |
| Poloxamer 188 | 100 | 9.25 |
| PEG 8000 | 200 | 18.52 |
| [Pyr1]-Apelin-13 | 180 | 16.67 |
| TOTAL | 1080 | 100.00% |

Methods of making inhalation apelin formulations and liposomes described herein are known in the art. In some embodiments, the liposomes are made by admixing a poloxamer, DSPC, DPPC, a PEG, and optionally cholesterol, to form a lipid film. The apelin peptide is then slowly added to the lipid film, thereby forming the liposomes. See, e.g., International application publication WO2018075822, which is hereby incorporated herein in its entirety.

In some embodiments, provided herein is a method of preventing or treating a coronavirus-associated disease in an individual, the method comprising administering to the individual an effective amount of an intranasal apelin formulation comprising an apelin peptide, wherein the liposomes in the inhalation apelin formulations comprise a weight percentage of the apelin peptide of between about 15.7% and 17.7%, a weight percentage of the poloxamer (e.g., poloxamer 188) of between about 8.3% and 10.3%, a weight percentage of DSPC of between about 27% and about 29%, a weight percentage of DPPC of between about 27% and about 29%, and a weight percentage of the PEG (e.g., PEG 8000) of between about 17.5% and about 19.5%, and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an intranasal apelin formulation comprising an apelin peptide, wherein the liposomes in the intranasal apelin formulations comprise a weight percentage of the apelin peptide of about 16.67%, a weight percentage of poloxamer 188 of about 9.25%, a weight percentage of DSPC of about 27.77%, a weight percentage of DPPC of about 27.77%, and a weight percentage of PEG 8000 of about 18.52%, and wherein the amount of the apelin peptide in the intranasal formulation is about 3 mg to about 5 mg, such as any of about 3.5 mg, 4 mg, or 4.5 mg, per 100 kg of body weight of the individual. In some embodiments, the intranasal apelin formulation is administered twice per week. In some embodiments, the apelin peptide is pyroglutamyl apelin-13 ([Pyrl]-apelin-13). In some embodiments, the coronavirus-associated disease is COVID-19.

iii. Devices for Delivery of an Aerosolized Apelin Formulation and Methods of Delivering an Aerosolized Apelin Formulation

In some aspects, the present disclosure provides devices for delivery of an aerosolized apelin formulation described herein, and methods of delivery of an aerosolized apelin formulation to an individual in need thereof.

In some embodiments, provided herein is a device for delivery of an apelin formulation, wherein the device contains the apelin formulation, and wherein the device is configured to aerosolize the apelin formulation.

In some embodiments, the apelin formulation comprises caffeine, benzalkonium chloride, and an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, caffeine and benzalkonium chloride are associated with and/or encapsulated in the liposome. In some embodiments, the device is a nasal sprayer.

In some embodiments, the apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG). In some embodiments, the device is an inhaler. In some embodiments, the device is configured to provide the aerosolized apelin formulation to a ventilator.

In some embodiments, provided herein is a method of delivering an aerosolized apelin formulation to an individual, the method comprising using a device described herein to administer the aerosolized apelin formulation to the individual in need thereof.

### 2. Parenteral Formulations of Apelin for Parenteral Administration

In some embodiments, provided herein is a method of preventing or treating a coronavirus-associated disease in an individual, the method comprising administering to the individual an effective amount of a parenteral apelin formulation comprising an apelin peptide. In some embodiments, the method comprises administering the parenteral apelin formulation to the individual via subcutaneous administration. In some embodiments, the method comprises administering the parenteral apelin formulation to the individual via intravenous administration.

In some embodiments, the effective amount of the apelin peptide in the parenteral apelin formulation is about 1 mg to about 10 mg, such as any of about 1 mg to about 8 mg, about 1 mg to about 5 mg, about 3 mg to about 8 mg, about 2 mg to about 6 mg, or about 3 mg to about 5 mg, per 100 kg of body weight of the individual. In some embodiments, the effective amount of the apelin peptide in the parenteral apelin formulation is at least about 0.5 mg, such as at least about any of 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, per 100 kg of body weight of the individual. In some embodiments, the effective amount of the apelin peptide in the parenteral apelin formulation is less than about 10 mg, such as less than about any of 9.5 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.5 mg, 2 mg, 1.5 mg, or 1 mg, per 100 kg of body weight of the individual. In some embodiments, the effective amount of the apelin peptide in the parenteral apelin formulation is about any of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, per 100 kg of body weight of the individual.

In some embodiments, the parenteral apelin formulation comprises an effective amount of the apelin peptide that is at least partially encapsulated in a liposome. In some embodiments, the liposomes comprise a lipid bilayer encapsulating a liquid core. In some embodiments, wherein each liposome comprises a plurality of the apelin peptide, a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the liposomes are configured such that the apelin peptide presented on the outer surface of the liposomes may associate with, such as bind, a relevant receptor and/or target binding site. In some embodiments, the parenteral apelin formulation comprises an amount of an apelin peptide that is not associated with or encapsulated in a liposome.

In some embodiments, the liposomes comprises a liquid core comprising a second subset of the plurality of the apelin peptide. In some embodiments, the liposomes are configured to hold a certain concentration, or range thereof, of the second subset of the apelin peptide.

The parenteral apelin formulation can be administered according to the discretion of the practicing physician. In some embodiments, the parenteral apelin formulation is administered daily. In some embodiments, the parenteral apelin formulation is administered one or more, such as any of 2, 3, 4, 5, or 6, times per week. In some embodiments, the parenteral apelin formulation is administered twice per week. In some embodiments, the parenteral apelin formulation is administered weekly.

Administration of parenteral formulations are known in the art, and include intravenous (IV) administration, subcutaneous (SubQ) administration, intramuscular (IM) administration, and intradermal (ID) administration.

In some embodiments, the apelin formulation is suitable for parenteral administration. In some embodiments, the parenteral apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

The parenteral apelin formulations described herein comprise an apelin peptide. In some embodiments, the weight percentages of apelin in the parenteral apelin formulation is about 5% to about 55%, such as any of about 15% to about 35%, about 20% to about 30%, about 35% to about 55%, or about 40% to about 50%. In some embodiments, the weight percentages of apelin in the parenteral apelin formulation is at least about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%. In some embodiments, the weight percentages of apelin in the parenteral apelin formulation is at less than about any 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 19%, 18%, 17%, 16%, 15%, 14%, 13%, 12%, 11%, 10%, 9%, 8%, 7%, 6%, or 5%. In some embodiments, the weight percentages of apelin in the parenteral apelin formulation is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%.

The liposomes in the parenteral apelin formulation described herein comprise at least one poloxamer. In some embodiments, the at least one poloxamer is one or more of poloxamer 188, poloxamer 124, poloxamer 181, poloxamer 184, poloxamer 331, and poloxamer 407, or any combination thereof. In some embodiments, the poloxamer is poloxamer 188.

In some embodiments, the weight percentage of the poloxamer in the liposome is between about 1% and about 20%, such as between any of about 1% and about 18%, about 2% and about 14%, about 5% and about 11%, about 8% and about 9%, about 7.3% and about 9.3%, about 10% and about 20%, about 17.5% and about 22.5%, or about 14% and about 16%. In some embodiments, the weight percentage of the poloxamer in the liposome is at least about 1%, such as at least about any of 2%, 3%, 4%, 5%, 6%, 7%, 8%, 9%, 10%, 110%, 12%, 13%, 14%, 15%, 16%, 17%, 18%, 19%, or 20%. In some embodiments, the weight percentage of the poloxamer in the liposome is about 20% or less, such as about any of 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less. In some embodiments, the weight percentage of the poloxamer in the liposome is about any of 1%, 2%, 3%, 4%, 5%, 6%, 7%, 8%, 8.3%, 9%, 10%, 11%, 12%, 13%, 14%, 15%, 16%, 17%, 18%, 19%, 20%, 21%, 22%, 23%, 24%, or 25%.

The liposomes in the parenteral apelin formulation described herein comprise 1,2-distearoyl-sn-glycero-3-phosphocholine (DSPC). In some embodiments, the weight percentage of DSPC in liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DSPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DSPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DSPC in the liposome is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The liposomes in the parenteral apelin formulation described herein comprise 2-dipalmitoyl-sn-glycero-3-phosphocholine (DPPC). In some embodiments, the weight percentage of DPPC in the liposome is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DPPC in the liposome is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DPPC in the liposome is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DPPC in the liposome is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The liposomes in the parenteral apelin formulation described herein comprise polyethylene glycol (PEG). In some embodiments, the PEG has an average molecular weight of between about 200 to about 20,000 Daltons. In some embodiments, the PEG is PEG 200, PEG 300, PEG 400, PEG 1000, PEG 1540, PEG 4000, PEG 5000, PEG 6000, PEG 7000, PEG 8000, PEG 9000, or PEG 10000. In some embodiments, the PEG is PEG 8000.

In some embodiments, the weight percentage of the PEG in the liposome is between about 10% and about 20%, such as between any of about 12.5% and about 17.5%, about 14% and about 16%, about 15.6% and about 17.6%. In some embodiments, the weight percentage of the PEG in the liposome is at least about 10%, such as at least about any of 11%, 12%, 13%, 14%, 15%, 16%, 17%, 18%, 19%, or 20%. In some embodiments, the weight percentage of the PEG in the liposome is about 20% or less, such as about any of 19% or less, 18% or less, 17% or less, 16% or less, 15% or less, 14% or less, 13% or less, 12% or less, 11% or less, or 10% or less. In some embodiments, the weight percentage of the PEG in the liposome is about any of 10%, 11%, 12%, 13%, 14%, 15%, 16%, 16.6%, 17%, 18%, 19%, or 20%.

The liposomes in the parenteral apelin formulation described herein may optionally comprise cholesterol. In some embodiments, the weight percentage of cholesterol in the liposome is between about 0.1% and about 10%, such as between any of about 2.5% and about 7.5%, or about 4% and about 6%.

In some embodiments, the weight percentage of cholesterol in the liposome is at least about 0.1%, such as at least about any of 1%, 2%, 3%, 4%, 5%, 6%, 7%, 8%, 9%, or 10%. In some embodiments, the weight percentage of cholesterol in the liposome is about 10% or less, such as about any of 9% or less, 8% or less, 7% or less, 6% or less, 5% or less, 4% or less, 3% or less, 2% or less, or 1% or less. In some embodiments, the weight percentage of cholesterol in the liposome is about any of 1%, 2%, 3%, 4%, 5%, 6%, 7%, 8%, 9%, or 10%.

In some embodiments, the liposomes in the parenteral apelin formulation herein comprise a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%.

In some embodiments, the liposomes in the parenteral apelin formulation herein comprise a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

In some embodiments, the liposomes in the parenteral apelin formulation herein comprise a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%.

In some embodiments, the liposomes in the parenteral apelin formulation herein comprise a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

In some embodiments, the lipid-based nanoparticle is a liposome prepared according to Formulation 3 (Table 4). In some embodiments, the lipid-based nanoparticle is a liposome prepared according to Formulation 4 (Table 4).

TABLE 4
Formulation 3 and Formulation 4.

| | Formulation 3 | | Formulation 4 | |
|---|---|---|---|---|
| | Weight (mg) | Weight % | Weight (mg) | Weight % |
| DSPC | 450 | 25.00% | 100 | 10.00% |
| DPPC | 450 | 25.00% | 100 | 10.00% |
| Poloxamer 188 | 150 | 8.33% | 150 | 15.00% |
| PEG 8000 | 300 | 16.67% | 150 | 15.00% |
| [Pyr1]-Apelin-13 | 450 | 25.00% | 450 | 45.00% |
| Cholesterol | — | — | 50 | 5.00% |
| TOTAL | 1800 | 100.00% | 1000 | 100.00% |

Methods of making thee apelin formulations and liposomes described in this section of the disclosure are known in the art. In some embodiments, the liposomes are made by admixing a poloxamer, DSPC, DPPC, a PEG, and optionally cholesterol, to form a lipid film. The apelin peptide is then slowly added to the lipid film, thereby forming the lipid-based nanoparticles. See, e.g., International application publication WO2018075822, which is hereby incorporated herein in its entirety.

In some embodiments, provided herein is a method of preventing or treating a coronavirus-associated disease in an individual, the method comprising administering to the individual an effective amount of an parenteral apelin formulation comprising an apelin peptide, wherein the parenteral apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG), wherein the liposomes comprise a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%, and wherein the amount of the apelin peptide in the parenteral formulation is about 3 mg to about 5 mg, such as any of about 3.5 mg, 4 mg, or 4.5 mg, per 100 kg of body weight of the individual. In some embodiments, the parenteral apelin formulation is administered via intravenous administration. In some embodiments, the parenteral apelin formulation is administered via subcutaneous administration. In some embodiments, the parenteral apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an parenteral apelin formulation comprising an apelin peptide, wherein the parenteral apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG), wherein the liposomes comprise a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%, and wherein the amount of the apelin peptide in the parenteral formulation is about 3 mg to about 5 mg, such as any of about 3.5 mg, 4 mg, or 4.5 mg, per 100 kg of body weight of the individual. In some embodiments, the parenteral apelin formulation is administered via intravenous administration. In some embodiments, the parenteral apelin formulation is administered via subcutaneous administration. In some embodiments, the parenteral apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an parenteral apelin formulation comprising an apelin peptide, wherein the parenteral apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), a polyethylene glycol (PEG), and cholesterol, wherein the liposomes comprise a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%, and wherein the amount of the apelin peptide in the parenteral formulation is about 3 mg to about 5 mg, such as any of about 3.5 mg, 4 mg, or 4.5 mg, per 100 kg of body weight of the individual. In some embodiments, the parenteral apelin formulation is administered via intravenous administration. In some embodiments, the parenteral apelin formulation is administered via subcutaneous administration. In some embodiments, the parenteral apelin formulation is administered twice per week. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an parenteral apelin formulation comprising an apelin peptide, wherein the parenteral apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), a polyethylene glycol (PEG), and cholesterol, wherein the liposomes comprise a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%, and wherein the amount of the apelin peptide in the parenteral formulation is about 3 mg to about 5 mg, such as any of about 3.5 mg, 4 mg, or 4.5 mg, per 100 kg of body weight of the individual. In some embodiments, the parenteral apelin formulation is administered via intravenous administration. In some embodiments, the parenteral apelin formulation is administered via subcutaneous administration. In some embodiments, the parenteral apelin formulation is administered twice per week. In some embodiments, the apelin peptide is pyroglutamyl apelin-13 ([Pyrl]-apelin-13). In some embodiments, the coronavirus-associated disease is COVID-19.

### 3. Oral Administration and Oral Formulations of Apelin

In some embodiments, provided herein is a method of preventing or treating a coronavirus-associated disease in an individual, the method comprising administering to the individual an effective amount of an oral apelin formulation comprising an apelin peptide. Also provided herein is an oral apelin formulation and dosage forms thereof.

In some embodiments, the effective amount of the apelin peptide in the oral apelin formulation is about 1 mg to about 10 mg, such as any of about 1 mg to about 8 mg, about 1 mg to about 6 mg, about 3 mg to about 8 mg, about 2 mg to about 6 mg, or about 4 mg to about 6 mg. In some embodiments, the effective amount of the apelin peptide in the oral apelin formulation is at least about 0.5 mg, such as at least about any of 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg. In some embodiments, the effective amount of the apelin peptide in the oral apelin formulation is less than about 10 mg, such as less than about any of 9.5 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.5 mg, 2 mg, 1.5 mg, or 1 mg. In some embodiments, the effective amount of the apelin peptide in the oral apelin formulation is about any of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg.

The oral apelin formulation can be administered according to the discretion of the practicing physician. In some embodiments, the oral apelin formulation is administered daily. In some embodiments, the oral apelin formulation is administered one or more, such as any of 2, 3, 4, 5, or 6, times per week. In some embodiments, the oral apelin formulation is administered twice per week. In some embodiments, the oral apelin formulation is administered weekly.

In some embodiments, the apelin formulation is suitable for oral administration. In some embodiments, the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the effective amount of the apelin peptide, a poloxamer, 1,2-distearoyl-sn-glycero-3-phosphocholine (DSPC), and 2-dipalmitoyl-sn-glycero-3-phosphocholine (DPPC).

In some embodiments, each of the plurality of lipid-based nanoparticles are not individually encapsulated by the carbohydrate matrix. In some embodiments, one or more of the lipid-based nanoparticles are not completely encapsulated by the carbohydrate matrix. In some embodiments, the carbohydrate matrix is not a surface coating on a lipid-based nanoparticle.

In some embodiments, the weight percentage of the carbohydrate matrix comprising the polysaccharide relative to the polysaccharide, the cross-linking agent, the apelin peptide, the poloxamer, DSPC, DPPC, and optionally the PEG and/or cholesterol (included in the weight percentage calculation when present), is about any of 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%.

In some embodiments, the weight percentage of the cross-linking agent relative to the polysaccharide, the cross-linking agent, the apelin peptide, the poloxamer, DSPC, DPPC, and optionally the PEG and/or cholesterol (included in the weight percentage calculation when present), is about any of 1%, 2%, 3%, 4%, or 5%.

In some embodiments, the weight percentage of the apelin active peptide, the poloxamer, DSPC, DPPC, and optionally the PEG and/or cholesterol (included in the weight percentage calculation when present), of the lipid-based nanoparticles relative to the polysaccharide, the cross-linking agent, the apelin active peptide, the poloxamer, DSPC, DPPC, and optionally the PEG and/or cholesterol (included in the weight percentage calculation when present), is about any of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%.

In some embodiments, the total amount of the apelin peptide in the oral apelin formulation is based on the amount of a lipid-based nanoparticle relative to a carbohydrate matrix comprising a polysaccharide and a cross-linking agent. For example, in some embodiments, an oral apelin formulation having a relatively low amount of the apelin peptide comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 98%, the weight percentage of the cross-linking agent is about 1%, and the weight percentage of the lipid-based nanoparticle is about 1%. In some embodiments, an oral apelin formulation having a relatively high amount of an apelin peptide comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 50%, the weight percentage of the cross-linking agent is about 1%, and the weight percentage of the lipid-based nanoparticle is about 49%. In some embodiments, an oral apelin formulation having a relatively high amount of an apelin peptide and a cross-linking agent comprises weight percentages of the non-solvent components in the carbohydrate matrix comprising the polysaccharide, the cross-linking agent, and the lipid-based nanoparticles as follows: the weight percentage of the carbohydrate matrix comprising the polysaccharide is about 48%, the weight percentage of the cross-linking agent is about 5%, and the weight percentage of the lipid-based nanoparticle is about 47%. In some embodiments, the weight percentage of the carbohydrate matrix comprising the polysaccharide is larger than the weight percentage of the lipid-based nanoparticles.

In some aspects, provided herein is an oral apelin formulation produced using a spraying, such as spray-drying, technique or a microemulsion technique described herein.

The oral apelin formulations described herein provide enhanced bioavailability of the apelin peptide therein, such as compared to when the apelin peptide is administered in any of the following ways: alone, in a lipid-based nanoparticle not embedded in a carbohydrate matrix comprising a polysaccharide, and in a carbohydrate matrix comprising a polysaccharide without a lipid-based nanoparticle. In some embodiments, when the oral apelin formulation is administered to an individual, such as a human, the apelin peptide has a bioavailability in an individual of about 1% or greater, such as about any of 1.1% or greater, 1.2% or greater, 1.3% or greater, 1.4% or greater, 1.5% or greater, 1.6% or greater, 1.7% or greater, 1.8% or greater, 1.9% or greater, 2% or greater, 2.1% or greater, 2.2% or greater, 2.3% or greater, 2.4% or greater, 2.5% or greater, 2.6% or greater, 2.7% or greater, 2.8% or greater, 2.9% or greater, 3% or greater, 3.1% or greater, 3.2% or greater, 3.3% or greater, 3.4% or greater, 3.5% or greater, 3.6% or greater, 3.7% or greater, 3.8% or greater, 3.9% or greater, 4% or greater, 4.1% or greater, 4.2% or greater, 4.3% or greater, 4.4% or greater, 4.5% or greater, 4.6% or greater, 4.7% or greater, 4.8% or greater, 4.9% or greater, 5% or greater, 6% or greater, 7% or greater, 8% or greater, 9% or greater, or 10% or greater.

In some embodiments, the oral apelin formulation is in a state that maintains the structure of the components described herein, e.g., a particle comprising a carbohydrate matrix comprising a polysaccharide, a cross-linking agent, and a plurality of lipid-based nanoparticles embedded in the carbohydrate matrix. In some embodiments, the oral apelin formulation is in a state that is suitable for oral administration. In some embodiments, the oral apelin formulation is in a state suitable for use in an oral dosage form. In some embodiments, the oral apelin formulation is a dried formulation, such as a dried powder.

The oral apelin formulations described herein comprise a plurality of lipid-based nanoparticles embedded in a carbohydrate matrix comprising a polysaccharide. In some embodiments, the lipid-based nanoparticles comprise the apelin peptide, a poloxamer, DSPC, and DPPC. In some embodiments, the lipid-based nanoparticles comprise a polyethylene glycol (PEG) and/or cholesterol. In some embodiments, the lipid-based nanoparticle is a liposome.

The oral apelin formulations described herein comprise an apelin peptide. In some embodiments, the weight percentages of apelin in the oral apelin formulation is about 5% to about 55%, such as any of about 15% to about 35%, about 20% to about 30%, about 35% to about 55%, or about 40% to about 50%. In some embodiments, the weight percentages of apelin in the oral apelin formulation is at least about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%. In some embodiments, the weight percentages of apelin in the oral apelin formulation is at less than about any 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 19%, 18%, 17%, 16%, 15%, 14%, 13%, 12%, 11%, 10%, 9%, 8%, 7%, 6%, or 5%. In some embodiments, the weight percentages of apelin in the oral apelin formulation is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, or 55%.

The lipid-based nanoparticles, such as liposomes, in the oral apelin formulation comprise a poloxamer. In some embodiments, the poloxamer is poloxamer 188, poloxamer 124, poloxamer 181, poloxamer 184, poloxamer 331, and poloxamer 407, or any combination thereof. In some embodiments, the poloxamer is poloxamer 188.

In some embodiments, the weight percentage of the poloxamer in the lipid-based nanoparticle is between about 1% and about 25%, such as between any of about 1% and about 20%, about 2% and about 14%, about 5% and about 11%, about 8% and about 9%, about 7.3% and about 9.3%, about 10% and about 20%, about 17.5% and about 22.5%, or about 14% and about 16%. In some embodiments, the weight percentage of the poloxamer in the lipid-based nanoparticles is at least about 1%, such as at least about any of 2%, 3%, 4%, 5%, 6%, 7%, 8%, 9%, 10%, 11%, 12%, 13%, 14%, 15%, 16%, 17%, 18%, 19%, or 20%. In some embodiments, the weight percentage of the poloxamer in the lipid-based nanoparticles is about 20% or less, such as about any of 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less. In some embodiments, the weight percentage of the poloxamer in the lipid-based nanoparticles is about any of 1%, 2%, 3%, 4%, 5%, 6%, 7%, 8%, 8.3%, 9%, 10%, 11%, 12%, 13%, 14%, 15%, 16%, 17%, 18%, 19%, 20%, 21%, 22%, 23%, 24%, or 25%.

The lipid-based nanoparticles, such as liposomes, in the oral apelin formulation comprise 1,2-distearoyl-sn-glycero-3-phosphocholine (DSPC). In some embodiments, the weight percentage of DSPC in the lipid-based nanoparticles is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DSPC in the lipid-based nanoparticles is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DSPC in the lipid-based nanoparticles is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DSPC in the lipid-based nanoparticles is about any of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

The lipid-based nanoparticles, such as liposomes, in the oral apelin formulation comprise 2-dipalmitoyl-sn-glycero-3-phosphocholine (DPPC). In some embodiments, the weight percentage of DPPC in the lipid-based nanoparticles is between about 5% and about 30%, such as between any of 5% and about 15%, about 7.5% and about 12.5%, about 9% and about 11%, about 20% and about 30%, about 22.5% and about 27.5%, or about 24% and about 26%.

In some embodiments, the weight percentage of DPPC in the lipid-based nanoparticles is at least about 5%, such as at least about any of 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the weight percentage of DPPC in the lipid-based nanoparticles is about 30% or less, such as about any of 29% or less, 28% or less, 27% or less, 26% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less. In some embodiments, the weight percentage of DPPC in the lipid-based nanoparticles is about any of 5%, 6%, 7%, 8%, 9%, 10%, 11%, 12%, 13%, 14%, 15%, 16%, 17%, 18%, 9%, 20%, 21%, 22%, 23%, 24%, 25%, 26%, 27%, 28%, 29%, or 30%.

In some embodiments, the lipid-based nanoparticles, such as liposomes, in the oral apelin formulation comprise a PEG. In some embodiments, the PEG has an average molecular weight of between about 200 to about 20,000 Daltons. In some embodiments, the PEG is PEG 200, PEG 300, PEG 400, PEG 1000, PEG 1540, PEG 4000, PEG 5000, PEG 6000, PEG 7000, PEG 8000, PEG 9000, or PEG 10000. In some embodiments, the PEG is PEG 8000.

In some embodiments, the weight percentage of the PEG in the lipid-based nanoparticles is between about 10% and about 20%, such as between any of about 12.5% and about 17.5%, about 14% and about 16%, about 15.6% and about 17.6%. In some embodiments, the weight percentage of the PEG in the lipid-based nanoparticles is at least about 10%, such as at least about any of 11%, 12%, 13%, 14%, 15%, 16%, 17%, 18%, 19%, or 20%. In some embodiments, the weight percentage of the PEG in the lipid-based nanoparticles is about 20% or less, such as about any of 19% or less, 18% or less, 17% or less, 16% or less, 15% or less, 14% or less, 13% or less, 12% or less, 11% or less, or 10% or less. In some embodiments, the weight percentage of the PEG in the lipid-based nanoparticles is about any of 10%, 11%, 12%, 13%, 14%, 15%, 16%, 16.6%, 17%, 18%, 19%, or 20%.

In some embodiments, the lipid-based nanoparticles, such as liposomes, in the oral apelin formulation comprise cholesterol. In some embodiments, the weight percentage of cholesterol in the lipid-based nanoparticles is between about 0.1% and about 10%, such as between any of about 2.5% and about 7.5%, or about 4% and about 6%.

In some embodiments, the weight percentage of cholesterol in the lipid-based nanoparticles is at least about 0.1%, such as at least about any of 1%, 2%, 3%, 4%, 5%, 6%, 7%, 8%, 9%, or 10%. In some embodiments, the weight percentage of cholesterol in the lipid-based nanoparticles is about 10% or less, such as about any of 9% or less, 8% or less, 7% or less, 6% or less, 5% or less, 4% or less, 3% or less, 2% or less, or 1% or less. In some embodiments, the weight percentage of cholesterol in the lipid-based nanoparticles is about any of 1%, 2%, 3%, 4%, 5%, 6%, 7%, 8%, 9%, or 10%.

In some embodiments, the lipid-based nanoparticles are liposomes comprising a lipid bilayer encapsulating a liquid core. In some embodiments, wherein each liposome comprises a plurality of the apelin peptide, a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge. In some embodiments, the lipid-based nanoparticle, such as a liposome, is configured such that the apelin peptide presented on the outer surface of the lipid-based nanoparticle may associate with, such as bind, a relevant receptor and/or target binding site.

In some embodiments, the lipid-based nanoparticle comprises a liquid core comprising a second subset of the plurality of the apelin peptide. In some embodiments, the lipid-based nanoparticle, such as a liposome, is configured to hold a certain concentration, or range thereof, of the second subset of the apelin peptide.

In some embodiments, the weight percentage of the apelin peptide in the lipid-based nanoparticles is between about 1% and about 70%, such as between any of about 5% and about 60%, about 15% and about 60%, about 15% and about 35%, about 20% and about 30%, about 22.5% and about 27.5%, about 24% and about 26%, about 35% and about 55%, about 40% and about 50%, about 42.5% and about 47.5%, or about 44% and about 46%. In some embodiments, the weight percentage of the apelin peptide in the lipid-based nanoparticles is at least about 15%, such as at least about any of 20%, 25%, 30%, 35%, 40%, 45%, 50%, 55%, 60%, or 65%. In some embodiments, the weight percentage of the apelin peptide in the lipid-based nanoparticles is about 70% or less, such as about any of 65% or less, 60% or less, 55% or less, 50% or less, 45% or less, 40% or less, 35% or less, 30% or less, 25% or less, 20% or less, 15% or less, or 10% or less. In some embodiments, the weight percentage of the apelin peptide in the lipid-based nanoparticles is about any of 5%, 10%, 15%, 20%, 25%, 30%, 35%, 40%, 45%, 50%, 55%, 60%, 65%, or 70%.

In some embodiments, the lipid-based nanoparticles, such as liposome, in the oral apelin formulation comprise a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%.

In some embodiments, the lipid-based nanoparticles, such as liposome, in the oral apelin formulation comprise a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%.

In some embodiments, the lipid-based nanoparticles, such as liposome, in the oral apelin formulation comprise a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%.

In some embodiments, the lipid-based nanoparticles, such as liposome, in the oral apelin formulation comprise a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%.

In some embodiments, the lipid-based nanoparticle in the oral apelin formulation is prepared according to Formulation 5 (Table 5). In some embodiments, the lipid-based nanoparticle in the oral apelin formulation is prepared according to Formulation 6 (Table 5).

TABLE 5
Formulation 5 and Formulation 6.

| | Formulation 5 | | Formulation 6 | |
|---|---|---|---|---|
| | Weight (mg) | Weight % | Weight (mg) | Weight % |
| DSPC | 450 | 25.00% | 100 | 10.00% |
| DPPC | 450 | 25.00% | 100 | 10.00% |
| Poloxamer 188 | 150 | 8.33% | 150 | 15.00% |
| PEG 8000 | 300 | 16.67% | 150 | 15.00% |
| [Pyr1]-Apelin-13 | 450 | 25.00% | 450 | 45.00% |
| Cholesterol | — | — | 50 | 5.00% |
| TOTAL | 1800 | 100.00% | 1000 | 100.00% |

Methods of making the oral apelin formulations, dosage forms, and lipid-based nanoparticles, such as liposomes, comprising an apelin peptide embedded therein are known in the art. In some embodiments, the lipid-based nanoparticles, such as liposomes, are made by admixing a poloxamer, DSPC, DPPC, and optionally a PEG and/or cholesterol, to form a lipid film. The apelin peptide is then slowly added to the lipid film, thereby forming the lipid-based nanoparticles. See, e.g., International application publication WO2018075822, which is hereby incorporated herein in its entirety.

The oral apelin formulations described herein comprise a plurality of particles comprising a carbohydrate matrix comprising a polysaccharide. In some embodiments, the size range of the plurality of particles is between about 1 μm and about 40 μm, such as between any of about 1 μm and about 10 μm, about 1 μm and about 20 μm, about 1 μm and about 30 μm, about 5 μm and about 25 μm, about 5 μm and about 35 μm, about 10 μm and about 40 μm, about 20 μm and about 40 μm, about 30 μm and about 40 μm, or about 20 μm and about 30 μm. In some embodiments, the average size of the plurality of particles is at least about 1 μm, such as at least about any of 2 μm, 3 μm, 4 μm, 5 μm, 10 μm, 15 μm, 20 μm, 25 μm, 30 μm, 40 μm, 45 μm, 50 μm, 55 μm, 60 μm, 65 μm, 70 μm, 75 μm, 80 μm, 85 μm, 90 μm, 95 μm, or 100 μm. In some embodiments, the average size of the plurality of particles is about 100 μm or less, such as about any of 95 μm or less, 90 μm or less, 85 μm or less, 80 μm or less, 75 μm or less, 70 μm or less, 65 μm or less, 60 μm or less, 55 μm or less, 50 μm or less, 45 μm or less, 40 μm or less, 35 μm or less, 30 μm or less, 25 μm or less, 20 μm or less, 15 μm or less, 10 μm or less, 5 μm or less, 4 μm or less, 3 μm or less, 2 μm or less, or 1 μm or less. In some embodiments, the size of the plurality of particles is homogenous. In some embodiments, the size of the plurality of particles is heterogeneous. In some embodiments, the size of the particle is as measured by dynamic light scattering.

In some embodiments, the plurality of particles are produced via a spray drying technique and/or milling technique.

In some embodiments, each of the plurality of particles comprises a plurality of pores. In some embodiments, the porosity of each of the plurality of particles is configured to adjust the amount of a lipid-based nanoparticle embedded therein.

In some embodiments, the polysaccharide is a pectin, gara gum, oak milk carbohydrate, banana carbohydrate, or any combination thereof. In some embodiments, the polysaccharide is a pectin. In some embodiments, the pectin is a citrus peel pectin. In some embodiments, the pectin is 150-grade pectin. In some embodiments, the pectin has a degree of esterification below about any of 70%, 65%, 60%, 55%, 50%, 45%, 40%, 35%, 30%, 25%, 20%, 15%, or 10%. In some embodiments, the degree of esterification of the pectin is selected based on a desired degree of cross-linking of the plurality of particles.

In some embodiments, the plurality of particles is not a gel or hydrogel.

In some embodiments, the carbohydrate matrix further comprises one or more of resveratrol, curcumin, and carnitine.

The oral formulations described herein comprise a plurality of particles comprising a cross-linking agent. In some embodiments, the cross-linking agent is a non-covalent cross-linking agent. In some embodiments, the cross-linking agent is a covalent cross-linking agent (e.g., generates one or more covalent linkages in a component, and/or between components, of the oral formulation).

In some embodiments, the cross-linking agent forms intra-particle crosslinks between portions of the carbohydrate matrix (e.g., between the polysaccharide). In some embodiments, the cross-linking agent forms intra-particle crosslinks between a portion of the carbohydrate matrix and a portion of the lipid-based nanoparticle (e.g., the apelin peptide). In some embodiments, the cross-linking agent forms inter-particle crosslinks.

In some embodiments, the cross-linking agent is selected from a divalent or polyvalent cation. In some embodiments, the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof. In some embodiments, the cross-linking agent is Ca²⁺. In some embodiments, the cross-linking agent is from a composition capable of generating Ca²⁺, such as CaCl₂). In some embodiments, the cross-linking agent is Zn²⁺. In some embodiments, the cross-linking agent is from a composition capable of generating Zn²⁺, such as ZnSO₄.

In some aspects, provided herein are oral dosage forms comprising an oral apelin formulation described herein. In some embodiments, the oral dosage form comprises more than one oral apelin formulation described herein, wherein each oral formulation is unique from the others in the oral dosage form, e.g., each has a different amount of an apelin peptide and/or a different weight percentage of the carbohydrate matrix.

In some embodiments, the oral dosage form comprises between about 0.01 mg and about 1 mg, such as between any of about 0.015 mg and about 0.1 mg, about 0.02 mg and about 0.03 mg, about 0.02 and about 0.1 mg, about 0.1 mg and about 0.5 mg, and about 0.5 mg and about 0.75 mg, of the apelin peptide. In some embodiments, the oral dosage form comprises at least about 0.01 mg, such as at least about any of 0.025 mg, 0.05 mg, 0.075 mg, 0.1 mg, 0.2 mg, 0.3 mg, 0.4 mg, 0.5 mg, 0.6 mg, 0.7 mg, 0.8 mg, 0.9 mg, or 1 mg, of the apelin peptide. In some embodiments, the oral dosage form comprises about any of the following amounts of the apelin peptide: 0.01 mg, 0.025 mg, 0.05 mg, 0.075 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, or 1 mg.

In some embodiments, the oral dosage form is a tablet, capsule, or caplet. In some embodiments, the oral dosage form comprises a vegetable- or gelatin-based capsule. In some embodiments, the oral dosage form comprises an oral formulation in a state suitable for oral administration. In some embodiments, the oral formulation in the oral dosage form is in a dried form, a semi-liquid form (such as a gel), or a liquid form (such as a suspension, solution, or emulsion).

In some embodiments, the oral dosage form further comprises a pharmaceutically acceptable excipient, pharmaceutically acceptable salt, diluent, carrier, vehicle, bulking agent, other inactive agents used to formulate oral dosage forms, or any combination thereof. Vehicles and excipients commonly employed in oral dosage forms include, for example, talc, gum Arabic, lactose, starch, magnesium stearate, cocoa butter, and paraffin derivatives. In some embodiments, the oral dosage form further comprises a preservative and/or a stabilizer. In some embodiments, the oral dosage form further comprises a cryoprotectant agent.

In some embodiments, the oral drug dosage form further comprises one or more of resveratrol, curcumin, and carnitine.

In some aspects, provided herein are methods of making the oral formulations and oral dosage forms described herein.

In some embodiments, the method of making the oral apelin formulation comprises admixing the carbohydrate matrix comprising the polysaccharide, the cross-linking agent, the apelin peptide, the poloxamer, DSPC, and DPPC, thereby obtaining the oral formulation. In some embodiments, the method further comprises admixing the PEG and/or cholesterol with the carbohydrate matrix comprising the polysaccharide, the cross-linking agent, the apelin peptide, the poloxamer, DSPC, and DPPC. In some embodiments, the method of making the oral apelin formulation comprises admixing pre-determined weight percentages of the carbohydrate matrix, the lipid-based nanoparticle, and the cross-linking agent, wherein: (i) the weight percentage of the carbohydrate matrix comprising the polysaccharide relative to the polysaccharide, the cross-linking agent, the apelin peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 48% to about 98%; (ii) the weight percentage of the cross-linking agent relative to the polysaccharide, the cross-linking agent, the apelin peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to about 5%; and (iii) the weight percentage of the apelin peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), of the lipid-based nanoparticles relative to the polysaccharide, the cross-linking agent, the apelin peptide, the poloxamer, 1,2-distearoyl-sn-glycero-3-phosphocholine (DSPC), 2-dipalmitoyl-sn-glycero-3-phosphocholine (DPPC), and optionally the PEG and/or cholesterol (included in the weight percentage calculation when present), is about 1% to 49%.

In some embodiments, the method of making the oral apelin formulation comprises use of a lipid-based nanoparticle, such as a liposome, comprising a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%. In some embodiments, the method of making the oral apelin formulation comprises use of a lipid-based nanoparticle, such as a liposome, comprising a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%.

In some embodiments, the oral apelin formulation is prepared using a spraying, such as spray-drying, technique or a microemulsion technique.

In some embodiments, provided herein is a method of making an oral apelin formulation described herein, the method comprising: (a) dissolving an amount of a material comprising a polysaccharide; (b) admixing an apelin peptide and a poloxamer in the dissolved material comprising the polysaccharide; (c) spray drying the solution resulting from step (b); and (d) suspending the particles produced from step (c) in a solution of DSPC, DPPC, and a cross-linking agent (and optionally a PEG and/or cholesterol), thereby making the oral apelin formulation.

In some embodiments, provided herein is a method of making an oral apelin formulation described herein, the method comprising: (a) obtaining a solution comprising a plurality of lipid-based nanoparticles; and (b) admixing the lipid-based nanoparticle solution with a carbohydrate matrix comprising a polysaccharide, wherein the admixing is performed at a temperature of about 40° C. to about 80° C., thereby making the oral apelin formulation. In some embodiments, the admixing is performed by spraying the lipid-based nanoparticle solution into the carbohydrate matrix. In some embodiments, the lipid-based nanoparticle solution comprises a cross-linking agent. In some embodiments, the method further comprises admixing the carbohydrate matrix embedded with lipid-based nanoparticles and a cross-linking agent.

In some embodiments, provided herein is a method of making an oral apelin formulation described herein, the method comprising: (a) dissolving an amount of a material comprising a polysaccharide; (b) admixing the apelin peptide and the poloxamer in the dissolved material comprising the polysaccharide; (c) forming an emulsion of the solution resulting from step (b); and (d) admixing the emulsion from step (c) with a solution of DSPC, DPPC, and a cross-linking agent (and optionally a PEG and/or cholesterol), thereby making the oral apelin formulation.

In some embodiments, the oral dosage form is produced by packaging an amount of an oral apelin formulation described herein in a suitable oral dosage form vehicle, such as a vegetable- or gelatin-based capsule. In some embodiments, the amount of the oral apelin formulation packaged in a suitable oral dosage form vehicle is based on the desired amount of the apelin peptide per oral dosage form.

In some embodiments, provided herein is a method of preventing or treating a coronavirus-associated disease in an individual, the method comprising administering to the individual an effective amount of an oral apelin formulation comprising an apelin peptide, wherein the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, wherein the lipid-based nanoparticle comprises the effective amount of the apelin peptide, a poloxamer, 1,2-distearoyl-sn-glycero-3-phosphocholine (DSPC), 2-dipalmitoyl-sn-glycero-3-phosphocholine (DPPC), and PEG, wherein the lipid-based nanoparticles, such as liposome, comprise a weight percentage of the apelin peptide of between about 23% and 27%, a weight percentage of the poloxamer (e.g., poloxamer 188) of between about 6.3% and 10.3%, a weight percentage of DSPC of between about 23% and about 27%, a weight percentage of DPPC of between about 23% and about 27%, and a weight percentage of the PEG (e.g., PEG 8000) of between about 14.7% and about 18.7%, and wherein the amount of the apelin peptide in the oral formulation is about 4 mg to about 6 mg, such as any of about 4.5 mg, 5 mg, or 5.5 mg. In some embodiments, the oral apelin formulation is orally administered daily. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an oral apelin formulation comprising an apelin peptide, wherein the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, wherein the lipid-based nanoparticle comprises the effective amount of the apelin peptide, a poloxamer, 1,2-distearoyl-sn-glycero-3-phosphocholine (DSPC), 2-dipalmitoyl-sn-glycero-3-phosphocholine (DPPC), and PEG, wherein the lipid-based nanoparticles, such as liposome, comprise a weight percentage of the apelin peptide of about 25%, a weight percentage of poloxamer 188 of about 8.3%, a weight percentage of DSPC of about 25%, a weight percentage of DPPC of about 25%, and a weight percentage of PEG 8000 of about 16.7%, and wherein the amount of the apelin peptide in the oral formulation is about 4 mg to about 6 mg, such as any of about 4.5 mg, 5 mg, or 5.5 mg. In some embodiments, the oral apelin formulation is orally administered daily. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an oral apelin formulation comprising an apelin peptide, wherein the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the effective amount of the apelin peptide, a poloxamer, 1,2-distearoyl-sn-glycero-3-phosphocholine (DSPC), 2-dipalmitoyl-sn-glycero-3-phosphocholine (DPPC), PEG, and cholesterol, wherein the lipid-based nanoparticles, such as liposome, comprise a weight percentage of the apelin peptide of between about 43% and 47%, a weight percentage of the poloxamer (e.g., poloxamer 188) of between about 13% and about 17%, a weight percentage of DSPC of between about 8% and about 12%, a weight percentage of DPPC of between about 8% and about 12%, a weight percentage of the PEG (e.g., PEG 8000) of between about 13% and about 17%, and weight percentage of cholesterol of between about 3% and about 7%, and wherein the amount of the apelin peptide in the oral formulation is about 4 mg to about 6 mg, such as any of about 4.5 mg, 5 mg, or 5.5 mg. In some embodiments, the oral apelin formulation is orally administered daily. In some embodiments, the apelin peptide is pyroglutamyl apelin-13 ([Pyrl]-apelin-13). In some embodiments, the coronavirus-associated disease is COVID-19.

In some embodiments, provided herein is a method of preventing or treating a coronavirus-associated disease in an individual, the method comprising administering to the individual an effective amount of an oral apelin formulation comprising an apelin peptide, wherein the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, wherein the lipid-based nanoparticle comprises the effective amount of the apelin peptide, a poloxamer, 1,2-distearoyl-sn-glycero-3-phosphocholine (DSPC), 2-dipalmitoyl-sn-glycero-3-phosphocholine (DPPC), PEG, and cholesterol, wherein the lipid-based nanoparticles, such as liposome, in the oral apelin formulation comprise a weight percentage of the apelin peptide of about 45%, a weight percentage of poloxamer 188 of about 15%, a weight percentage of DSPC of about 10%, a weight percentage of DPPC of about 10%, a weight percentage of PEG 8000 of about 15%, and weight percentage of cholesterol of about 5%, and wherein the amount of the apelin peptide in the oral formulation is about 4 mg to about 6 mg, such as any of about 4.5 mg, 5 mg, or 5.5 mg. In some embodiments, the oral apelin formulation is orally administered daily. In some embodiments, the apelin peptide is pyroglutamyl apelin-13 ([Pyrl]-apelin-13). In some embodiments, the coronavirus-associated disease is COVID-19.

### 4. Combination Treatments

In some aspects, provided herein are methods of treating a coronavirus-associated disease in an individual, the method comprising administering to the individual (i) an effective amount of an apelin formulation comprising an apelin peptide described herein, and (ii) another therapeutic agent. In some embodiments, the method comprises administering two or more other therapeutic agents. In some embodiments, the effective amount of the apelin formulation is administered in conjunction or in combination with the other therapeutic agent. In some embodiments, the effective amount of the apelin formulation is administered simultaneously with the other therapeutic agent. In some embodiments, the effective amount of the apelin formulation is administered sequentially with the other therapeutic agent. In some embodiments, the effective amount of the apelin formulation is administered concurrenlty with the other therapeutic agent.

In some embodiments, the other therapeutic agent is an anti-viral agent. In some embodiments, the anti-viral agent is selected from the group consisting of remdesivir, lopinavir/ritonavir, IFN-α, lopinavir, ritonavir, penciclovir, galidesivir, disulfiram, darunavir, cobicistat, ASC09F, disulfiram, nafamostat, griffithsin, alisporivir, chloroquine, nitazoxanide, baloxavir marboxil, oseltamivir, zanamivir, peramivir, amantadine, rimantadine, favipiravir, laninamivir, ribavirin, umifenovir, and any combination thereof. In some embodiments, the anti-viral agent is chloroquine. In some embodiments, the anti-viral agent is remdesivir. In some embodiments, the anti-viral agent is oseltamivir.

In some embodiments, the other therapeutic agent is selected from the group consisting of resveratrol, curcumin, and carnitine.

### EXEMPLARY EMBODIMENTS

Embodiment 1. A method of preventing or treating a coronavirus-associated disease in an individual, the method comprising administering to the individual an effective amount of an apelin formulation comprising an apelin peptide.

Embodiment 2. The method of embodiment 1, wherein the coronavirus-associated disease is Coronavirus Disease-2019 (COVID-19), an Angiotensin-Converting Enzyme 2 (ACE2)-associated disease, Acute Respiratory Distress Syndrome (ARDS), Severe Acute Respiratory Syndrome (SARS), or Middle East respiratory syndrome (MERS).

Embodiment 3. The method of embodiment 1 or 2, wherein the coronavirus-associated disease causes one or more of heart failure, lung failure, or sepsis-like conditions.

Embodiment 4. The method of any one of embodiments 1-3, wherein the coronavirus-associated disease is caused by a virus of the Coronaviridae family.

Embodiment 5. The method of embodiment 4, wherein the virus is of the Betacoronavirus genus.

Embodiment 6. The method of embodiment 4 or 5, wherein the virus is of the Sarbecovirus subgenus.

Embodiment 7. The method of any one of embodiments 4-6, wherein the virus is of the SARSr-CoV species.

Embodiment 8. The method of any one of embodiments 4-7, wherein the virus is a SARS-CoV strain.

Embodiment 9. The method of embodiment 4, wherein the virus is selected from the group consisting of Severe Acute Respiratory Syndrome coronavirus 2 (SARS-CoV-2), Severe Acute Respiratory Syndrome coronavirus (SARS-CoV), Bat SARS-like coronavirus WIV1 (Bat SL-CoV-WIV1), alpha coronaviruses 229E (HCoV-229E), New Haven coronavirus NL63 (HCoV-NL63), beta coronaviruses OC43 (HCoV-OC43), coronavirus HKU1 (HCoV-HKU1), and Middle East Respiratory Syndrome coronavirus (MERS-CoV).

Embodiment 10. The method of any one of embodiments 1-9, wherein the method further comprises administering another therapeutic agent.

Embodiment 11. The method of embodiment 10, wherein the other therapeutic agent is an anti-viral agent.

Embodiment 12. The method of embodiment 11, wherein the anti-viral agent is selected from the group consisting of remdesivir, lopinavir/ritonavir, IFN-α, lopinavir, ritonavir, penciclovir, galidesivir, disulfiram, darunavir, cobicistat, ASC09F, nafamostat, griffithsin, alisporivir, chloroquine, nitazoxanide, baloxavir marboxil, oseltamivir, zanamivir, peramivir, amantadine, rimantadine, favipiravir, laninamivir, ribavirin, umifenovir, and any combination thereof.

Embodiment 13. The method of any one of embodiments 1-12, wherein the apelin formulation is an aerosolized formulation.

Embodiment 14. The method of embodiment 13, wherein the apelin formulation is an intranasal apelin formulation.

Embodiment 15. The method of embodiment 14, wherein the apelin formulation is administered to the individual via intranasal administration.

Embodiment 16. The method of embodiment 14 or 15, wherein the apelin formulation comprises caffeine, benzalkonium chloride, and an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

Embodiment 17. The method of embodiment 13, wherein the apelin formulation is an inhalation apelin formulation.

Embodiment 18. The method of embodiment 17, wherein the apelin formulation is administered to the individual via inhalation administration.

Embodiment 19. The method of embodiment 17 or 18, wherein the apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

Embodiment 20. The method of embodiment 16 or 19, wherein the effective amount of the apelin peptide in the apelin formulation is about 1 mg to about 10 mg per 100 kg of body weight of the individual.

Embodiment 21. The method of any one of embodiments 13-20, wherein the apelin formulation is administered twice per week.

Embodiment 22. The method of any one of embodiments 1-12, wherein the apelin formulation is a parenteral formulation.

Embodiment 23. The method of embodiment 22, wherein the apelin formulation is administered to the individual via subcutaneous or intravenous administration.

Embodiment 24. The method of embodiment 22 or 23, wherein the apelin formulation comprises an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

Embodiment 25. The method of embodiment 24, wherein the apelin formulation further comprises cholesterol.

Embodiment 26. The method of embodiment 25, wherein the weight percentage of cholesterol in the liposome is about 0.1% to about 10%.

Embodiment 27. The method of any one of embodiments 24-26, wherein the effective amount of the apelin peptide in the apelin formulation is about 1 mg to about 10 mg per 100 kg of body weight of the individual.

Embodiment 28. The method of any one of embodiments 22-27, wherein the apelin formulation is administered twice per week.

Embodiment 29. The method of any one of embodiments 16-28, wherein the at least one poloxamer is poloxamer 188, poloxamer 124, poloxamer 181, poloxamer 184, poloxamer 331, and poloxamer 407, or any combination thereof.

Embodiment 30. The method of any one of embodiments 16-29, wherein the weight percentage of the poloxamer in the liposome is about 1% to about 20%.

Embodiment 31. The method of any one of embodiments 16-30, wherein the weight percentage of DSPC in the liposome is about 5 wt % and about 30 wt %.

Embodiment 32. The method of any one of embodiments 16-31, wherein the weight percentage of DPPC in the liposome is about 5 wt % and about 30 wt %.

Embodiment 33. The method of any one of embodiments 16-32, wherein the average molecular weight of the PEG is about 200 Da to about 20000 Da.

Embodiment 34. The method of any one of embodiments 16-33, wherein the average molecular weight of the PEG is about 8000 Da.

Embodiment 35. The method of any one of embodiments 16-34, wherein the weight percentage of the PEG in the liposome is about 10% to about 20%.

Embodiment 36. The method of any one of embodiments 16-35, wherein the apelin peptide is selected from the group consisting of apelin-12, apelin-13, pyroglutamyl apelin-13 ([Pyrl]-apelin-13), apelin-17, apelin-19, and apelin-36.

Embodiment 37. The method of any one of embodiments 16-36, wherein the weight percentage of the apelin peptide in the liposome is about 15 wt % to about 60 wt %.

Embodiment 38. The method of any one of embodiments 1-12, wherein the apelin formulation is an oral apelin formulation.

Embodiment 39. The method of embodiment 38, wherein the apelin formulation is administered via oral administration.

Embodiment 40. The method of embodiment 38 or 39, wherein the oral apelin formulation comprises a plurality of particles, wherein each particle comprises a carbohydrate matrix comprising a polysaccharide, a cross-linking agent, and a plurality of lipid-based nanoparticles embedded in the carbohydrate matrix, and wherein the lipid-based nanoparticle comprises the effective amount of the apelin peptide, a poloxamer, 1,2-distearoyl-sn-glycero-3-phosphocholine (DSPC), and 2-dipalmitoyl-sn-glycero-3-phosphocholine (DPPC).

Embodiment 41. The method of embodiment 40, wherein the lipid-based nanoparticles are liposomes comprising a lipid bilayer encapsulating a liquid core.

Embodiment 42. The method of embodiment 41, wherein each liposome comprises a plurality of the apelin peptide, wherein a first subset of the plurality of the apelin peptide is configured such that one portion of the apelin peptide is embedded in the lipid bilayer and another portion of the apelin peptide is presented on the outer surface of the lipid bilayer or the inner surface of the lipid bilayer facing the liquid core, wherein the portion of the apelin peptide embedded in the lipid bilayer is the stretch of amino acids having a net hydrophobic characteristic, and wherein the portion of the apelin peptide presented on the outer surface of the lipid bilayer or the inner surface of the lipid bilayer facing the liquid core is the stretch of amino acids having a net positive charge.

Embodiment 43. The method of embodiment 41 or 42, wherein the liquid core comprises a second subset of the plurality of the apelin peptide.

Embodiment 44. The method of any one of embodiments 40-43, wherein the effective amount of the apelin peptide in the apelin formulation is about 1 mg to about 10 mg.

Embodiment 45. The method of any one of embodiments 40-44, wherein the apelin peptide is selected from the group consisting of apelin-12, apelin-13, pyroglutamyl apelin-13 ([Pyrl]-apelin-13), apelin-17, apelin-19, and apelin-36.

Embodiment 46. The method of any one of embodiments 38-45, wherein the apelin formulation is administered to the individual daily.

Embodiment 47. The method of any one of embodiments 40-46, wherein the weight percentage of the apelin peptide in the lipid-based nanoparticles is about 15% to about 60%.

Embodiment 48. The method of any one of embodiments 40-47, wherein the poloxamer is poloxamer 188, poloxamer 124, poloxamer 181, poloxamer 184, poloxamer 331, and poloxamer 407, or any combination thereof.

Embodiment 49. The method of any one of embodiments 40-48, wherein the weight percentage of the poloxamer in the lipid-based nanoparticles is about 1% to about 20%.

Embodiment 50. The method of any one of embodiments 40-49, wherein the weight percentage of DSPC in the lipid-based nanoparticles is about 5% to about 30%.

Embodiment 51. The method of any one of embodiments 40-50, wherein the weight percentage of DPPC in the lipid-based nanoparticles is about 5% to about 30%.

Embodiment 52. The method of any one of embodiments 40-51, wherein the lipid-based nanoparticle further comprises a polyethylene glycol (PEG).

Embodiment 53. The method of embodiment 52, wherein the average molecular weight of the PEG is about 200 Da to about 20000 Da.

Embodiment 54. The oral formulation of embodiment 50 or 53, wherein the average molecular weight of the PEG is about 8000 Da.

Embodiment 55. The method of any one of embodiments 52-54, wherein the weight percentage of the PEG in the lipid-based nanoparticles is about 10% to about 20%.

Embodiment 56. The method of any one of embodiments 40-55, wherein the lipid-based nanoparticle further comprises cholesterol.

Embodiment 57. The method of embodiment 56, wherein the weight percentage of cholesterol in the lipid-based nanoparticles is about 0.1% to about 10%.

Embodiment 58. The method of any one of embodiments 40-57, wherein the size range of the plurality of particles is about 1 μm to about 40 μm.

Embodiment 59. The method of any one of embodiments 40-58, wherein each of the plurality of particles comprises a plurality of pores.

Embodiment 60. The method of any one of embodiments 40-59, wherein the polysaccharide is a pectin, gara gum, oak milk carbohydrate, or banana carbohydrate.

Embodiment 61. The method of embodiment 60, wherein the pectin is a citrus peel pectin.

Embodiment 62. The method of embodiment 60 or 61, wherein the pectin is 150-grade pectin.

Embodiment 63. The method of any one of embodiments 40-62, wherein the cross-linking agent is selected from a divalent or polyvalent cation.

Embodiment 64. The method of embodiment 63, wherein the divalent or polyvalent cation is selected from Ca²⁺, Zn²⁺, Pb²⁺, Cu²⁺, Ba²⁺, Sr²⁺, Cd⁺², Co²⁺, Ni²⁺, or a combination thereof.

Embodiment 65. The method of any one of embodiments 40-62, wherein the apelin peptide has a bioavailability in an individual of about 2% or greater.

Embodiment 66. An aerosolized apelin formulation comprising an effective amount of an apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

Embodiment 67. The aerosolized apelin formulation of embodiment 66, further comprising caffeine and/or benzalkonium chloride.

Embodiment 68. The aerosolized apelin formulation of embodiment 67, wherein the caffeine and/or benzalkonium chloride are associated with and/or encapsulated in the liposome.

Embodiment 69. A device for delivery of an aerosolized apelin formulation, wherein the device contains the aerosolized apelin formulation of any one of embodiments 66-68, and wherein the device is configured to aerosolize the apelin formulation.

Embodiment 70. The device of embodiment 69, wherein the device is a nasal sprayer.

Embodiment 71. The device of embodiment 69, wherein the device is an inhaler.

Embodiment 72. The device of embodiment 69, wherein the device is configured to provide the aerosolized apelin formulation to a ventilator.

Embodiment 73. A method of delivering an aerosolized apelin formulation to an individual, the method comprising using a device of any one of embodiments 69-72 to administer the aerosolized apelin formulation to the individual in need thereof.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates preparation of an apelin formulation comprising liposomes for aerosolized administration.

300 mg DSPC, 300 mg DPPC, 100 mg poloxamer, and 200 mg PEG 8000 were dissolved in chloroform and sonicated until the lipid constituents dissolved completely in the solvent. The solvent was removed under vacuum using rotovapor. The lipid film was dissolved in 300 mM citric acid and sonicated until the lipid cake was dissolved. The solution was then dialyzed against water and lyophilized. About 100 mg of the lipid cake was used for the apelin formulation. 20 mg of an apelin peptide was dissolved in 10 mL of ultrapure water and slowly added to the lipid cake and sonicated to form a uniform suspension. Caffeine at 0.4 mg/mL and Benzalkonium chloride at a concentration of 0.2 mg/mL were added and the solution was frozen and lyophilized. The lyophilized preparation was resuspended in water, flash frozen, and lyophilized. The resuspension and lyophilization were repeated twice. The final resuspension for animal administration was made in 1 mL sterile normal saline having 10 mg of liposome components, 2 mg of apelin, 0.2 mg caffeine, and 0.1 mg Benzalkonium chloride.

### Example 2

This example demonstrates preparation of liposomes comprising an apelin peptide, a poloxamer, a PEG, DSPC, and DPPC.

DSPC and DPPC were reconstituted in ethanol and sonicated until completely dissolved (the minimum amount of ethanol need to dissolve DSPC and DPPC was used). PEG 8000 and Poloxamer 188 were reconstituted in ethanol and sonicated until completely dissolved. The DSPC and DPPC solution was mixed with the PEG 800 and Poloxamer 188 solution in a single vial. Then, the mixed solution was subjected to nitrogen to remove the solvent. The final solid was dried in vacuum for 3 hours. The lipid film was dissolved in citric acid (300 mmol) solution. The film was suspended for 15 minutes and then filtered with a polycarbonate filter (0.2 nm size). The mixture was exchanged with distilled water by dialysis and then lyophilized. Apelin (180 mg) was then dissolved in distilled water and added to the lipid film. Additional water was added while slowly mixing the solution for about 30 minutes to 1 hour. The formed liposomes were then incubated at 37° C. for 90 minutes prior to lyophilization.

### Example 3

This example demonstrates preparation techniques for oral formulations of an apelin peptide comprising pectin, a poloxamer, DSPC, DPPC, and calcium chloride.

The oral formulation was prepared by a spray drying technique. 5 mg of pectin was weighed and dissolved in 100 mL of water by slow addition of pectin in small portions to a stirring solution of water. Stirring was continued overnight to obtain a viscous solution of 5% pectin. 200 mg of the apelin peptide and 2 g of the poloxamer were added to the pectin solution. The solution was diluted by adding 800 mL of water followed by adding 200 mL of ethanol. The solution was stirred to obtain a homogeneous solution. The solution was then spray dried using the following settings: an inlet temperature of 60° C., aspirator set to 90-95, and a condenser temperature set to 4° C. The accumulated particles were transferred in the collection vessel to a desiccator. 5 g of the particles were suspended in a solution containing 500 mg of DSPC, 500 mg of DPPC, and 200 mg of calcium chloride in acetone. The suspension was stirred overnight. Subsequently, the acetone was evaporated under vacuum using a rotavapor. The resulting formulation was used to produce a calculated amount of the dosage for animal administration. Particles were suspended in water just before the oral administration.

The oral formulation was prepared by a microemulsion technique. 5 mg pectin was weighed and dissolved in 100 mL of water by slow addition of pectin in small portions to a stirring solution of water. Stirring was continued overnight to obtain a viscous solution of 5% pectin. 200 mg of the apelin peptide and 2 g of the poloxamer were added to the pectin solution. The solution was diluted by adding 100 mL of water followed by adding 800 mL of dichloromethane (DCM). The solution was stirred to obtain an emulsion. The emulsion was added to a solution containing 500 mg of DSPC, 500 mg of DPPC, and 200 mg of calcium chloride in acetone. The suspension was stirred overnight. The solvent was evaporated under vacuum using a rotavapor. The resulting formulation was used to produce a calculated amount of the dosage for animal administration. Particles were suspended in water just before the oral administration.

### Example 4

This example demonstrates treatment of patients having a coronavirus-associate disease, such as COVID-19, with an apelin formulation described herein.

Patients that test positive for infection by SARS-CoV-2 or that are at risk of infection to SARS-CoV-2 are assigned to one of four treatment administration groups to receive one of the following: (1) an intranasal apelin formulation to be administered at 4 mg per 100 kg of person twice weekly; (2) an inhalation apelin formulation to be administered at 4 mg per 100 kg of person twice weekly; (3) an parenteral apelin formulation to be administered at 4 mg per 100 kg of person twice weekly; (4) an oral apelin formulation to be administered at 5 mg daily. Patients are monitored to assess for disease progression and/or infection. Dosing amount, frequency, and administration route may be adjusted as deemed necessary by a physician. Signs and symptoms of disease status, including cardio-pulmonary distress and sepsis-like conditions, are monitored during the administration period.

What is claimed is:

1. An aerosolized formulation of an apelin peptide comprising an effective amount of the apelin peptide that is at least partially encapsulated in a liposome comprising an amount of each of the following: at least one poloxamer, 1,2-distearoyl-sn-glycero-3-phosphocholine (DSPC), 1,2-dipalmitoyl-sn-glycero-3-phosphocholine (DPPC), and a polyethylene glycol (PEG).

2. The aerosolized formulation of claim 1, wherein the composition further comprises caffeine.

3. The aerosolized formulation of claim 1 or 2, wherein the composition further comprises benzalkonium chloride.

4. The aerosolized formulation of claim 2 or 3, wherein caffeine and/or benzalkonium chloride is associated with and/or encapsulated in the liposome.

5. The aerosolized formulation of any one of claims 1-4, wherein the at least one poloxamer is poloxamer 188, poloxamer 124, poloxamer 181, poloxamer 184, poloxamer 331, and poloxamer 407, or any combination thereof.

6. The aerosolized formulation of any one of claims 1-5, wherein the at least one poloxamer is poloxamer 188.

7. The aerosolized formulation of any one of claims 1-6, wherein the weight percentage of the at least poloxamer in the liposome is about 1% to about 20%.

8. The aerosolized formulation of any one of claims 1-7, wherein the weight percentage of DSPC in the liposome is about 5 wt % and about 30 wt %.

9. The aerosolized formulation of any one of claims 1-8, wherein the weight percentage of DPPC in the liposome is about 5 wt % and about 30 wt %.

10. The aerosolized formulation of any one of claims 1-9, wherein the average molecular weight of the PEG is about 200 Da to about 20000 Da.

11. The aerosolized formulation of any one of claims 1-10, wherein the average molecular weight of the PEG is about 8000 Da.

12. The aerosolized formulation of any one of claims 1-11, wherein the weight percentage of the PEG in the liposome is about 10% to about 20%.

13. The aerosolized formulation of any one of claims 1-12, wherein the apelin peptide is selected from the group consisting of apelin-12, apelin-13, pyroglutamyl apelin-13 ([Pyrl]-apelin-13), apelin-17, apelin-19, and apelin-36.

14. The aerosolized formulation of any one of claims 1-13, wherein the apelin peptide is [Pyrl]-apelin-13.

15. The aerosolized formulation of any one of claims 1-14, wherein the weight percentage of the apelin peptide in the liposome is about 15 wt % to about 60 wt %.

16. The aerosolized formulation of any one of claims 1-15, further comprising a pharmaceutically acceptable excipient.

17. The aerosolized formulation of any one of claims 1-16, wherein the liposome comprises a weight percentage of the apelin peptide of between about 15.7% and 17.7%, a weight percentage of the poloxamer of between about 8.3% and 10.3%, a weight percentage of DSPC of between about 27% and about 29%, a weight percentage of DPPC of between about 27% and about 29%, and a weight percentage of the PEG of between about 17.5% and about 19.5%.

18. The aerosolized formulation of any one of claims 1-16, wherein the liposome comprises a weight percentage of the apelin peptide of about 16.67%, a weight percentage of poloxamer 188 of about 9.25%, a weight percentage of DSPC of about 27.77%, a weight percentage of DPPC of about 27.77%, and a weight percentage of PEG 8000 of about 18.52%.

19. The aerosolized formulation of any one of claims 3-16, wherein the composition comprises a weight percentage of the apelin peptide of between about 15.5% and 17.5%, a weight percentage of the poloxamer of between about 8.3% and 10.3%, a weight percentage of DSPC of between about 27% and about 29%, a weight percentage of DPPC of between about 27% and about 29%, a weight percentage of the PEG of between about 17.5% and about 19.5%, a weight percentage of caffeine of between about 0.01% and 0.3%, and a weight percentage of benzalkonium chloride of about 0.00010% and 0.010%.

20. The aerosolized formulation of any one of claims 3-16, wherein the composition comprises a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.51%, a weight percentage of caffeine of about 0.019%, and a weight percentage of benzalkonium chloride of about 0.009%.

21. The aerosolized formulation of any one of claims 3-16, wherein the liposome comprises a weight percentage of the apelin peptide of between about 15.5% and 17.5%, a weight percentage of the poloxamer of between about 8.3% and 10.3%, a weight percentage of DSPC of between about 27% and about 29%, a weight percentage of DPPC of between about 27% and about 29%, a weight percentage of the PEG of between about 17.5% and about 19.5%, a weight percentage of caffeine of between about 0.01% and 0.3%, and a weight percentage of benzalkonium chloride of about 0.00010% and 0.010%.

22. The aerosolized formulation of any one of claims 3-16, wherein the liposome comprises a weight percentage of the apelin peptide of about 16.66%, a weight percentage of poloxamer 188 of about 9.26%, a weight percentage of DSPC of about 27.77%, a weight percentage of DPPC of about 27.77%, a weight percentage of PEG 8000 of about 18.51%, a weight percentage of caffeine of about 0.019%, and a weight percentage of benzalkonium chloride of about 0.009%.

23. The aerosolized formulation of any one of claims 1-22, wherein the aerosolized formulation is an intranasal apelin formulation.

24. The aerosolized formulation of any one of claims 1-22, wherein the aerosolized formulation is an inhalation apelin formulation.

25. A method of preparing the aerosolized formulation of any one of claims 1-24, comprising:
dissolving the DSPC and the DPPC in ethanol and sonicating until dissolved to form a first composition;
dissolving the PEG and the poloxamer in ethanol and sonicating until dissolved to form a second composition;
forming a lipid film from the first composition and the second composition; and
mixing the apelin peptide with the lipid film to form a liposome.

26. The method of claim 25, further comprising admixing caffeine and/or benzalkonium chloride with the material that forms the lipid film.

27. The method of claim 25, further comprising admixing caffeine and/or benzalkonium chloride with the liposome.

28. A device for delivery of an aerosolized apelin formulation, wherein the device contains an aerosolized formulation of any one of claims 1-24, and wherein the device is configured to aerosolize the composition.

29. The device of claim 28, wherein the device is a nasal sprayer.

30. The device of claim 28, wherein the device is an inhaler.

31. The device of claim 28, wherein the device is configured to provide the aerosolized apelin formulation to a ventilator.

32. A method of delivering an aerosolized apelin formulation to an individual, the method comprising using a device of any one of claims 28-31 to administer the aerosolized apelin formulation to the individual in need thereof.
